(19) United States
(12) Patent Application Publication
(10) Pub. No.: US 2010/0209513 A1
(43) Pub. Date: Aug. 19, 2010

(54) PHARMACEUTICAL COMPOSITION CONTAINING MICRONIZED PARTICLES OF NAPHTHOQUINONE-BASED COMPOUND

(76) Inventors: In Geun Jo, Chungcheongnam-do (KR); Sang-Ku Yoo, Gyeonggi-do (KR); Myung-gyu Park, Gyeonggi-do (KR); Taehwan Kwak, Gyeonggi-do (KR)

Correspondence Address: BIRCH STEWART KOLASCH & BIRCH, PO BOX 747, FALLS CHURCH, VA, 22040-0747, US

(21) Appl. No.: 12/682,504

(22) PCT Filed: Oct. 7, 2008
(86) PCT No.: PCT/KR08/05885

§ 371 Date: Apr. 9, 2010

(30) Foreign Application Priority Data
Oct. 11, 2007 (KR) 10-2007-0102463
Nov. 26, 2007 (KR) 10-2007-0121009

(51) Int. Cl. A61K 31/343; C07D 307/93; A61K 9/16; A61P 3/00; A61P 9/00; A61P 15/10; A61P 13/08; A61P 13/12; A61P 27/06

(52) U.S. Cl. 424/489; 549/457; 514/468; 977/773

(57) ABSTRACT

Provided is a pharmaceutical composition having excellent in vivo absorption properties by increasing solubility and absorption rate of a sparingly-soluble naphthoquinone-based compound via incorporation of micronized particles of a certain naphthoquinone-based compound.

### FIELD OF THE INVENTION

The present invention relates to a pharmaceutical composition comprising micronized particles of a naphthoquinone-based compound. More specifically, the present invention relates to a pharmaceutical composition having excellent in vivo absorption properties by increasing solubility and absorption rate of a sparingly-soluble naphthoquinone-based compound via incorporation of micronized particles of a certain naphthoquinone-based compound.

### BACKGROUND OF THE INVENTION

It is known that β-lapachone, as one of a naphthoquinone-based compound, and its derivatives can be used as anticancer, antibiotic and anti-fungal agent (U.S. Pat. No. 5,969,163, U.S. Pat. No. 5,824,700, U.S. Pat. No. 5,763,625, U.S. Pat. No. 5,641,773, U.S. Pat. No. 4,898,870 and U.S. Pat. No. 5,985,331), and similar compounds are recited in WO06/128120, WO04/045557, WO96/033988 WO94/004145, WO97/031936, which are known as anticancer drug.

The inventors of the present invention have synthesized 1,2-naphthoquinone-based compounds including tanshinone, and have ascertained that 1,2-naphthoquinone-based compounds including tanshinone are effective for prevention and treatment of metabolic diseases such as obesity, diabetes; restenosis, impotence, prostatic disease, hypertension, cardiac diseases, renal diseases and glaucoma (see, for example KR2004-0116339, KR2006-14541, PCT/KR2007/006012, PCT/KR2007/006013, PCT/KR2007/006011, KR 2007-0136105, KR 2007-0139740, KR2007-0141303).

However, the aforesaid naphthoquinone-based compounds are sparingly-soluble materials which are soluble at a low degree of about 2 to 10% only in high-solubility solvents, such as CH₂Cl₂, CHCl₃, CH₂ClCH₂Cl, CH₃CCl₃, Monoglyme, and Diglyme, but are poorly soluble in other ordinary polar or non-polar solvents. For this reason, the naphthoquinone-based compounds suffer from various difficulties associated with formulation of preparations for in vivo administration, in spite of their excellent pharmacological effects.

Therefore, when these drug compounds are administered by themselves or in the form of a conventional simple formulation via an oral route, there is substantially no absorption of the drug into the body, that is, the bioavailability of the drug is very low, so it is impossible to exert the intrinsic efficacy of the drug. Particularly, these drug compounds do not exert therapeutic effects until they are absorbed into the body in an amount exceeding a certain concentration. For these reasons, in order to sufficiently and satisfactorily exploit inherent pharmacological properties of these naphthoquinone-based compounds, there is an urgent need for development and introduction of a method which is capable of maximizing the bioavailability of these drugs.

In this connection, Boothman et al. suggested in U.S. Pat. No. 689,050 the technique that β-lapachon is contained within polymer such as β-cyclodextrin to thereby increase the solubility.

However, this technique has a problem that since a drug is contained within polymer, the amount of the drug is limited depending upon the amount of the polymer. Therefore, in the case of an oral preparation, particularly, the oral preparation of high dosage, the total amount of the oral preparation will be increased excessively, which limits the use of this oral preparation in some cases.

Also, the technique was suggested in WO06/20719 and WO06/20722 that naphthoquinone-based compounds are converted into a prodrug form by bonding with polymer or molecular such amino acid to thereby increase the solubility; however, this technique has the problem that the structure of the compound cannot be maintained.

Using of dispersants usually available for sparingly-soluble compounds was also considered as a method for increasing the solubility of naphthoquinone-based compounds, which required a large amount of dispersants, thus the above-mentioned problem is possibly caused in the case of the high dosage preparation.

### SUMMARY OF THE INVENTION

As a result of a variety of extensive and intensive studies and experiments to solve the problems as described above, the inventors of the present invention have discovered that a pharmaceutical composition comprising micronized particles of naphthoquinone-based compound, wherein the naphthoquinone-based compound is one or more selected from the compounds as represented by Formulas 1 and 2:

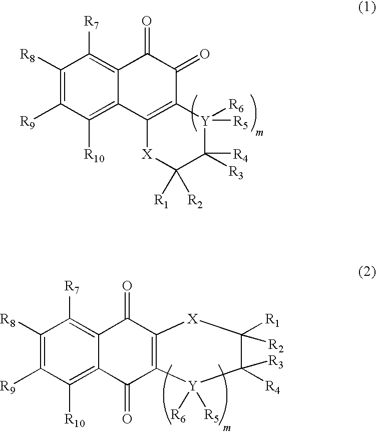

wherein

R₁ to R₆ are each independently hydrogen, hydroxyl, halogen, amino, alkylamino, dialkylamino, substituted or unsubstituted C₁-C₁₀alkyl, substituted or unsubstituted C₁-C₁₀alkenyl, substituted or unsubstituted C₁-C₁₀ alkynyl, substituted or unsubstituted C₁-C₁₀ alkoxy, substituted or unsubstituted C₁-C₁₀ alkoxycarbonyl, substituted or unsubstituted C₁-C₁₀ acyl, substituted or unsubstituted C₃-C₈ cycloalkyl, substituted or unsubstituted C₄-C₁₀ aryl, substituted or unsubstituted —(CH₂)ₙ-aryl, substituted or unsubstituted —(CH₂)ₙ-heterocyclic and substituted or unsubstituted —(CH₂)ₙ₋₁₀-phenyl, or two substituents thereof may be taken together to form a double bond, or substituted or unsubstituted C₃-C₆ cyclic structure which may be saturated or partially or completely unsaturated, wherein the substituent may be at least one selected from the group consisting of hydrogen, hydroxyl, C₁-C₁₀ alkyl, substituted or unsubstituted C₁-C₁₀ alkynyl, alkoxy, C₁-C₁₀ alkoxycarbonyl and C₁-C₁₀ alkylamino;

R₇ to R₁₀ are each independently hydrogen, hydroxyl, halogen, amino, alkylamino, dialkylamino, substituted or unsubstituted C₁-C₁₀ alkyl, substituted or unsubstituted C₁-C₁₀alkenyl, substituted or unsubstituted C₁-C₁₀ alkynyl, substituted or unsubstituted C₁-C₁₀ alkoxy, substituted or unsubstituted C₁-C₁₀ alkoxycarbonyl, substituted or unsubstituted C₁-C₁₀ acyl, substituted or unsubstituted C₄-C₁₀ aryl, and substituted or unsubstituted —(CH₂)ₙ₋₁₀-phenyl, or two substituents thereof may be taken together to form a double bond, or substituted or unsubstituted C₃-C₆ cyclic structure which may be saturated or partially or completely unsaturated, wherein the substituent may be at least one selected from the group consisting of hydrogen, hydroxyl, halogen, C₁-C₁₀ alkyl, C₁-C₁₀ alkenyl, C₁-C₁₀ alkynyl, C₁-C₁₀ alkoxy, C₁-C₁₀ alkoxycarbonyl, C₁-C₁₀ alkylamino, C₃-C₈ cycloalkyl, C₃-C₈ hetero cycloalkyl, C₄-C₁₀ aryl and C₄-C₁₀ heteroaryl;

X is O, S or NR′, wherein R′ is hydrogen or C₁-C₆ alkyl;

Y is C, S, N, or O, with proviso that when Y is S or O, R₅ and R₆ are nothing and when Y is N, R₅ is hydrogen or C₁-C₆ lower alkyl and R₆ is nothing; and

m is 0 or 1, with proviso that when m is 0, carbon atoms adjacent to m form a cyclic structure via a direct bond, and n is 0˜10 integer,

or a pharmaceutically acceptable salt, solvate or isomer thereof.

Preferably, X is O or S, and Y is C or O.

In one preferred example, R₁ to R₆ may be each independently selected from the group consisting of hydrogen, hydroxy, halogen, substituted or unsubstituted C₁-C₁₀ alkyl, substituted or unsubstituted alkenyl, substituted or unsubstituted C₁-C₁₀ alkoxy and —(CH₂)ₙ-phenyl, or R₁ and R₂ or R₂ and R₃ may be taken together to form a double bond, or substituted or unsubstituted C₃-C₆ cyclic structure, wherein the substituent may be hydrogen or C₁-C₁₀ alkyl.

In another preferred example, R₇ to R₁₀ may be each independently selected from the group consisting of hydrogen, hydroxyl, halogen, substituted or unsubstituted C₁-C₁₀ alkyl and substituted or unsubstituted C₁-C₁₀ alkoxy.

Among compounds of Formula 1 or 2 in accordance with the present invention, preferred are compounds represented by Formula 1-1 or 2-1 wherein X is O and Y is C, or compounds represented by Formula 1-2 or 2-2 wherein X is S.

Among compounds of the naphthoquinone-based compound, preferred are compounds of Formula 3 to 7 below.

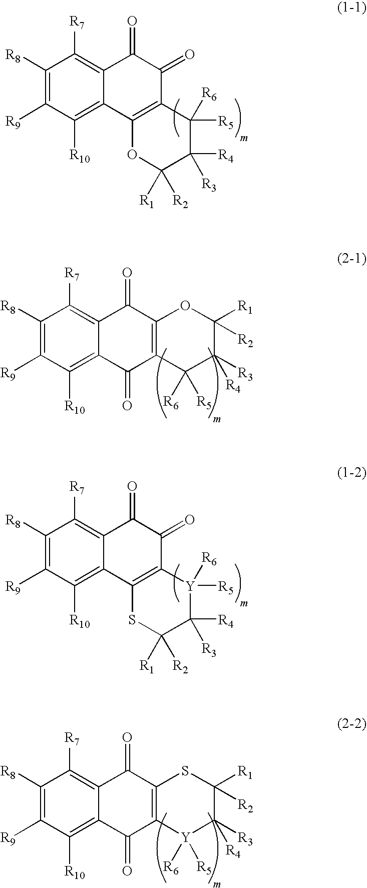

wherein, R₁ to R₁₀, Y and m are defined as in the Formula 1.

In another example, compounds of Formula 1 or 2 may be compounds of Formula 1-3 or 2-3 wherein m is 0, and adjacent carbon atoms form a cyclic structure (furan ring) via a direct bond therebetween are often referred to as “Furano-o-naphthoquinone derivatives” hereinafter.

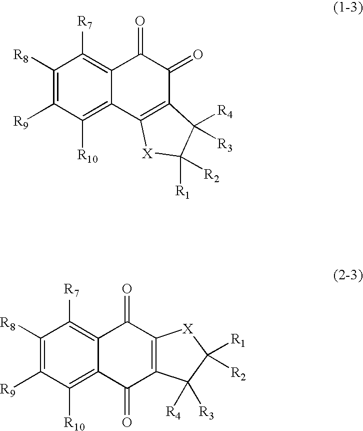

wherein, R₁ to R₁₀ and X are defined as the above.

Further, compounds of Formula 1 or Formula 2 may be compounds of Formula 1-4 or Formula 2-4 wherein m is 1 are often referred to as “Thiopyrano-1,2-naphthoquinone derivatives” hereinafter.

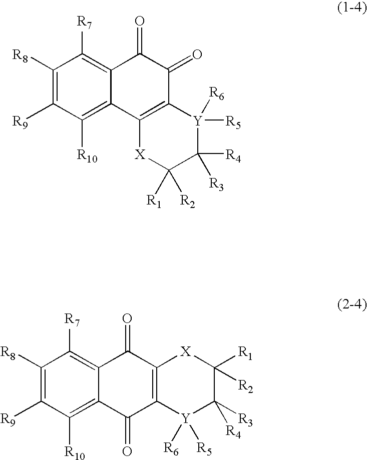

wherein, R₁ to R₁₈, X, Y and m are defined as the above.

In another example, compounds of Formula 1 which are R₇ and R₈ formed a cyclic structure via a direct bond therebetween may have structures of Formula 1-5 or 1-6, and compounds of Formula 1 may be compounds 2-5 or 2-6.

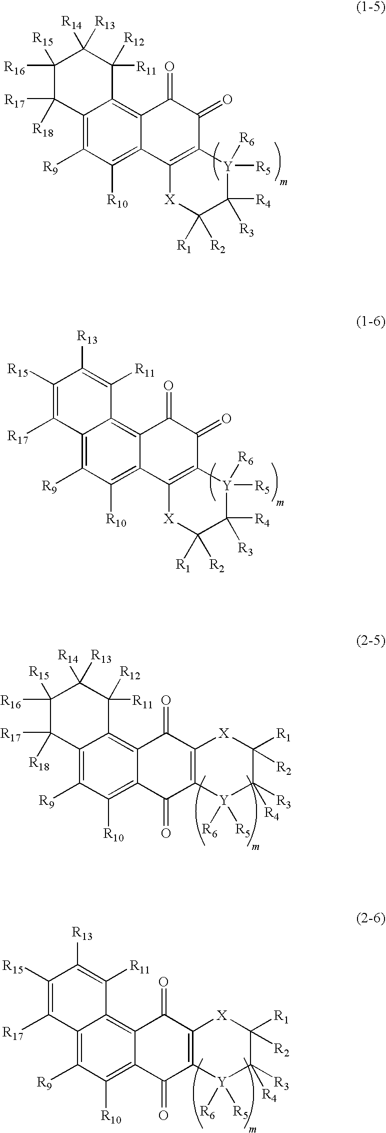

wherein, R₁ to R₁₈, X, Y and m are defined as the above. Preferably, R₁ to R₁₈ may be at least one selected from hydrogen, hydroxy, C₁-C₁₀ alkyl, C₁-C₁₀ alkenyl, C₁-C₁₀ alkynyl, C₁-C₁₀ alkoxy, C₃-C₈ cycloalkyl and phenyl, each independently.

In a pharmaceutical composition of the present invention, a scope of a naphthoquinone-based compound all include that a pharmaceutically acceptable salt, solvate or isomer thereof.

The term “pharmaceutical composition” as used herein means a mixture of a compound of Formula 1 or 2 with other chemical components, such as diluents or carriers. The pharmaceutical composition facilitates administration of the compound to an organism. Various techniques of administering a compound are known in the art and include, but are not limited to oral, injection, aerosol, parenteral and topical administrations. Pharmaceutical compositions can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

As used the present disclosure, the term “pharmaceutically acceptable salt” means a formulation of a compound that does not cause significant irritation to an organism to which it is administered and does not abrogate the biological activity and properties of the compound. Examples of the pharmaceutical salt may include acid addition salts of the compound with acids capable of forming a non-toxic acid addition salt containing pharmaceutically acceptable anions, for example, inorganic acids such as hydrochloric acid, sulfuric acid, nitric acid, phosphoric acid, hydrobromic acid and hydroiodic acid; organic carbonic acids such as tartaric acid, formic acid, citric acid, acetic acid, trichloroacetic acid, trifluoroacetic acid, gluconic acid, benzoic acid, lactic acid, fumaric acid, maleic acid and salicylic acid; or sulfonic acids such as methanesulfonic acid, ethanesulfonic acid, benzenesulfonic acid and p-toluenesulfonic acid. Specifically, examples of pharmaceutically acceptable carboxylic acid salts include salts with alkali metals or alkaline earth metals such as lithium, sodium, potassium, calcium and magnesium, salts with amino acids such as lysine, arginine, and guanidine, salts with organic bases such as dicyclohexylamine, N-methyl-D-glucamine, tris(hydroxymethyl)methylamine, diethanolamine, choline and triethylamine. The compound of Formula 1 in accordance with the present invention may be converted into salts thereof, by conventional methods well-known in the art.

As used herein, the term “solvate” means a compound of the present invention or a salt thereof, which further includes a stoichiometric or non-stoichiometric amount of a solvent bound thereto by non-covalent intermolecular forces. Preferred solvents are volatile, non-toxic, and/or acceptable for administration to humans. Where the solvent is water, the solvate refers to a hydrate.

As used herein, the term “isomer” means a compound of the present invention or a salt thereof that has the same chemical formula or molecular formula but is optically or sterically different therefrom.

Unless otherwise specified, the term “compound of Formula 1 or 2” or “naphthoquinone-based compound” is intended to encompass a compound per se, and a pharmaceutically acceptable salt, solvate and isomer thereof.

As used herein, the term “alkyl” refers to a radical which contains carbon and hydrogen, without unsaturation. The alkyl radical may be linear or branched. Examples of alkyl radical include, but are not limited to, methyl, ethyl, propyl, isopropyl, hexyl, t-butyl and sec-butyl.

Lower alkyl is C₁-C₁₀ alkyl (for example, the alkyl which has 1˜10 carbon atoms in its linear or branched alkyl mainchain). The alkyl can be substituted optionally. When substituted, the alkyl can be substituted as 4 and less substituents at any specific bonding point (at any carbon atom).

Meanwhile, when the alkyl is substituted by another alkyl group, it may be used as the same meaning of “branched alkyl”.

The term “alkenyl” means an unsaturated aliphatic group that is similar to alkyl mentioned above such as length and substitutability but contains one or more carbon-carbon double bonds.

For example, the term “alkenyl” includes linear alkenyl (for example, ethenyl, propenyl, butenyl, pentenyl, hexenyl, heptenyl, octenyl, nonenyl, decenyl), branched alkenyl, cycloalkenyl (alicyclic compound, for example, cyclopropenyl, cyclopentenyl, cyclohexenyl, cycloheptenyl, cyclooctenyl), cycloalkenyl which is substituted by alkyl or alkenyl and alkenyl which is substituted by cycloalkyl or cycloalkenyl. The term “alkenyl” also includes an additional alkenyl group which has one or more carbohydrogen mainchain carbon atoms substituted by oxygen, nitrogen, sulfur or phosphorus. For specific example, a linear or branched alkenyl group has 6 and less carbon atoms at its mainchain (for example, C₂-C₆ for linear, C₃-C₆ for branched). Similarly, cycloalkenyl may have 3˜8 carbon atoms in the ring system; more preferably, have 5˜6 carbon atoms.

The term “alkynyl” means an unsaturated aliphatic group that is similar to alkyl mentioned above such as length and substitutability but contains one or more carbon-carbon triple bonds. For example, the term “alkynyl” includes linear alkynyl (for example, ethynyl, propynyl, butynyl, pentynyl, hexynyl, heptynyl, octynyl, nonynyl, decynyl), branched alkynyl (including alkynyl which is substituted by alkyl or alkenyl) and alkynyl which is substituted by cycloalkyl or cycloalkenyl. The term “alkynyl” also includes an additional alkynyl group which has one or more carbohydrogen mainchain carbon atoms substituted by oxygen, nitrogen, sulfur or phosphorus. For specific example, a linear or branched alkynyl group has 6 and less carbon atoms at its mainchain (for example, C₂-C₆ for linear, C₃-C₆ for branched).

When the alkyl, the alkynyl or the alkenyl is substituted, they can be substituted by substituents such as hydroxyl, carboxylate, oxo, halogen (for example, F, Cl, Br, I), haloalkyl (for example, CCl₃ or CF₃), alkyloxycarbonyl (—C(O)R), alkylcarbonyloxy (—OCOR), carbamoyl (—NHCOOR— or —OCONHR—), urea (—NHCONHR—), thiol, cyano, nitro, amino, acylamino, C₁-C₆ alkylthio, arylthio, C₁-C₆ alkyl, C₁-C₆ alkoxy, aryloxy, alkylcarbonyloxy, arylcarbonyloxy, C₃-C₆ cycloalkyl, C₃-C₆ cycloalkyloxy, C₂-C₆ alkenyl, C₂-C₆ alkynyl, aryl, aminocarbonyl, C₁-C₆ alkylcarbonyl, C₃-C₆ cycloalkylcarbonyl, heterocyclylcarbonyl, arylcarbonyl, aryloxycarbonyl, C₁-C₆ alkoxycarbonyl, C₃-C₆ cycloalkyloxycarbonyl, heterocyclyloxycarbonyl, C₁-C₆ alkylsulfonyl, arylsulfonyl, heterocyclyl.

Preferably, the substituents are at least one selected from hydrogen, hydroxy, halogen, C₁-C₁₀ alkyl, C₂-C₁₀ alkenyl, substituted or unsubstituted alkynyl, C₁-C₁₀ alkoxy, C₁-C₁₀ alkoxycarbonyl, C₁-C₁₀ alkylamino, C₃-C₈ cycloalkyl, C₃-C₈ heterocycloalkyl, C₄-C₁₀ aryl and C₄-C₁₀ heteroaryl.

The term “cycloalkyl” means an alkyl group which includes 3˜15 carbon atoms, preferably, 3˜8 carbon atoms, without any alternate or resonance double bond. The cycloalkyl may include 1˜4 rings. Examples of the cycloalkyl include cyclopropyl, cyclobutyl, cyclopentyl, cyclohexyl and adamantly. An example substituent for cycloalkyl is halogen, C₁-C₁₀alkyl, C₁-C₁₀alkoxy, C₁-C₁₀alkylhydroxy, amino, nitro, cyano, thiol or C₁-C₁₀ alkylthio.

The term “heterocycloalkyl” means a carbocyclic group in which one or more ring carbon atoms are substituted with hetero atoms such as nitrogen(N), sulfur(S) or oxygen(O), and means saturated or unsaturated 7˜11 membered bicyclic heterocyclic rings or chemically stable non-aromatic 3˜8 membered monocyclic heterocyclic rings, and may form a additional ring via fusion, spiro, cross-linking. Each heterocyclic ring is composed of at least one carbon atom and 1˜4 hetero atoms. Heterocyclyl radical is bound with any endocyclic ring which creates a stable structure. Preferred heterocyclic ring includes, but are not limited to furan, thiophen, pyrrole, pyrroline, pyrrolidine, oxazole, thiazole, imidazole, imidazoline, imidazolidine, pyrazole, pyrazoline, pyrazolidine, isothiazole, triazole, thiadiazole, pyran, pyridine, piperidine, morpholine, thiomorpholine, pyridazine, pyrimidine, pyrazine, piperazine and triazine. Heterocyclic ring includes 3˜7 membered monocyclic heterocyclic rings such as, but not limited to, piperidinyl, pyranyl, piperazinyl, morpholinyl, thiamorpholinyl and tetrahydrofuranyl, more preferably 5˜7 membered monocyclic heterocyclic rings.

As used herein, the term “aryl” refers to an aromatic substituent group which has at least one ring having a conjugated pi (π) electron system and includes both carbocyclic aryl (for example, phenyl) and heterocyclic aryl (for example, pyridine) groups. This term includes monocyclic or fused-ring polycyclic (i.e., rings which share adjacent pairs of carbon atoms) groups. The aryl may be a carbocyclic compound or may have 1˜4 hetero atoms (for example, nitrogen (N), sulfur (S) or oxygen (O)) in its aromatic ring system optionally.

Examples of aryl or heteroaryl include, but are not limited to, phenyl, naftyl, pyridyl, pyrimidyl, pyrolyl, isothiazolyl, triazolyl, tetrazolyl, pyrazolyl, oxazolyl, isooxazolyl, pyrazinyl, pyridazinyl, triazinyl, quinazolinyl, thiazolyl, benzothiophenyl, furanyl, imidazolyl and thiophenyl.

The cycloalkyl, heterocycloalkyl, aryl and heteroaryl may be optionally substituted by substituents, but are not limited to, for example, hydroxyl, halogen, thiol, cyano, nitro, amino, acylamino, C₁-C₆ alkylthio, arylthio, C₁-C₆ alkyl, C₁-C₆ alkoxy, aryloxy, alkylcarbonyloxy, arylcarbonyloxy, C₃-C₆ cycloalkyl, C₃-C₆ cycloalkyloxy, C₂-C₆ alkenyl, C₂-C₆ alkynyl, aryl, carboxylate, aminocarbonyl, C₁-C₆ alkylcarbonyl, C₃-C₆ cycloalkylcarbonyl, heterocyclylcarbonyl, arylcarbonyl, aryloxycarbonyl, C₁-C₆ alkoxycarbonyl, C₃-C₆ cycloalkyloxycarbonyl, heterocyclyloxycarbonyl, aryloxycarbonyl, C₁-C₆ alkylsulfonyl, arylsulfonyl, and heterocyclyl.

The substituents R₁ to R₆ in Formula 1 or 2 in accordance with the present invention may be optionally substituted by substituent(s) which is(are) one or more selected from hydrogen, hydroxyl, halogen, C₁-C₁₀ alkyl, C₂-C₁₀ alkenyl, substituted or unsubstituted C₁-C₁₀ alkynyl, alkoxy, C₁-C₁₀ alkoxycarbonyl and C₁-C₁₀ alkylamino. Further, the substituents R₇ to R₁₀ may be also substituted, and the substituent(s) is(are) one or more selected from hydrogen, hydroxyl, halogen, C₁-C₁₀ alkyl, C₂-C₁₀ alkenyl, substituted or unsubstituted C₁-C₁₀ alkynyl, C₁-C₁₀ alkoxy, C₁-C₁₀ alkoxycarbonyl and C₁-C₁₀alkylamino, C₃-C₈ cycloalkyl, C₃-C₈heterocycloalkyl, C₄-C₁₀ aryl and C₄-C₁₀ heteroaryl.

The term “alkoxy” means —O— alkyl (wherein, alkyl is defined as the above). The alkoxy bind to mainchain, aryl or heteroaryl via oxo-bridge. The alkoxy may be linear or branched, but linear is preferred. For example, methoxy, ethyloxy, propoxy, butyloxy, t-butyloxy or i-propoxy is included. Preferred alkoxy includes 1˜4 carbon atoms; particularly preferred alkoxy includes 1˜3 carbon atoms.

The term “halogen” or “halo” includes VIIa group elements, for example, chlorine (Cl), bromine (Br), fluorine (F), iodine (I).

The term “amine” or “amino” includes compound which has a covalent coupled nitrogen atom with one or more carbon or hetero atoms.

Among compounds in accordance with the present invention, particularly preferred are compounds of Table 1 below, but are not limited to.

TABLE 1

| | | | |
|---|---|---|---|
| 1 | 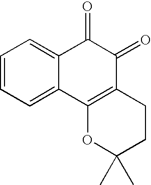 | C₁₅H₁₄O₃ | 242.27 |
| 2 | 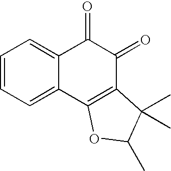 | C₁₅H₁₄O₃ | 242.27 |
| 3 | 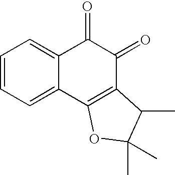 | C₁₅H₁₄O₃ | 242.27 |
| 4 | 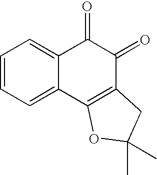 | C₁₄H₁₂O₃ | 228.24 |
| 5 | 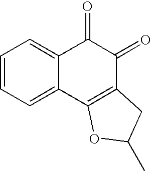 | C₁₃H₁₀O₃ | 214.22 |
| 6 | 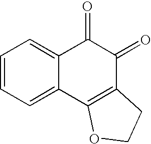 | C₁₂H₈O₃ | 200.19 |
| 7 | 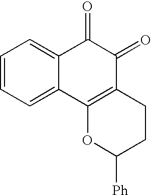 | C₁₉H₁₄O₃ | 290.31 |
| 8 | 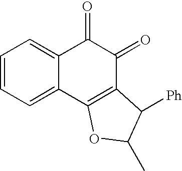 | C₁₉H₁₄O₃ | 290.31 |
| 9 | 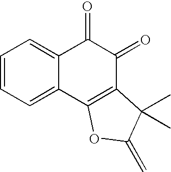 | C₁₅H₁₂O₃ | 240.25 |
| 10 | 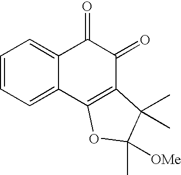 | C₁₆H₁₆O₄ | 272.30 |
| 11 | 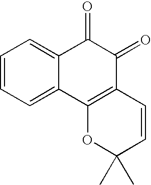 | C₁₅H₁₂O₃ | 240.25 |
| 12 | 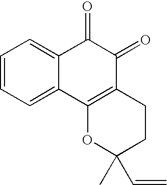 | C₁₆H₁₄O₃ | 254.28 |
| 13 | 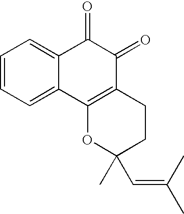 | C₁₈H₁₈O₃ | 282.33 |
| 14 | 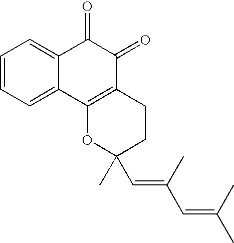 | C₂₁H₂₂O₃ | 322.40 |
| 15 | 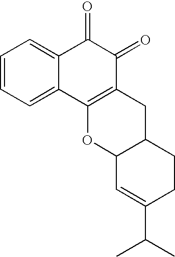 | C₂₁H₂₂O₃ | 322.40 |
| 16 | 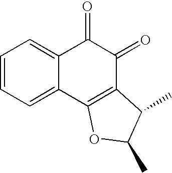 | C₁₄H₁₂O₃ | 228.24 |
| 17 | 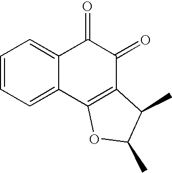 | C₁₄H₁₂O₃ | 228.24 |
| 18 | 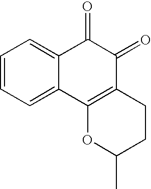 | C₁₄H₁₂O₃ | 228.24 |
| 19 | 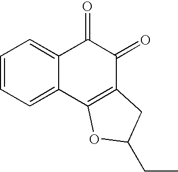 | C₁₄H₁₂O₃ | 228.24 |
| 20 | 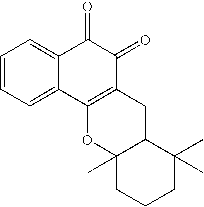 | C₂₀H₂₂O₃ | 310.39 |
| 21 | 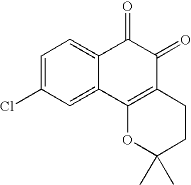 | C₁₅H₁₃ClO₃ | 276.71 |
| 22 | 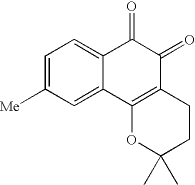 | C₁₆H₁₆O₃ | 256.30 |
| 23 | 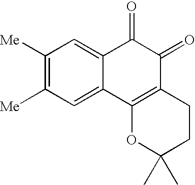 | C₁₇H₁₈O₅ | 302.32 |
| 24 | 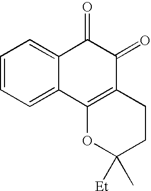 | C₁₆H₁₆O₃ | 256.30 |
| 25 | 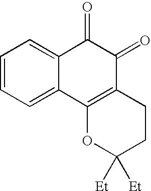 | C₁₇H₁₈O₃ | 270.32 |
| 26 | 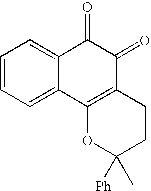 | C₂₀H₁₆O₃ | 304.34 |
| 27 | 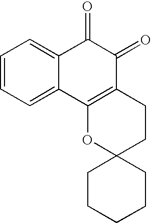 | C₁₈H₁₈O₃ | 282.33 |
| 28 | 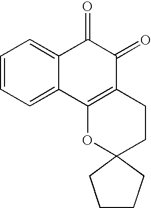 | C₁₇H₁₆O₃ | 268.31 |
| 29 | 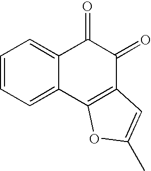 | C₁₃H₈O₃ | 212.20 |
| 30 | 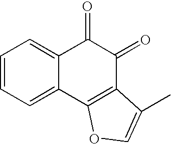 | C₁₃H₈O₃ | 212.20 |
| 31 | 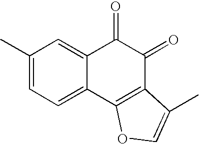 | C₁₄H₁₀O₃ | 226.23 |
| 32 | 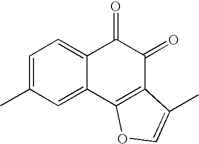 | C₁₄H₁₀O₃ | 226.23 |
| 33 | 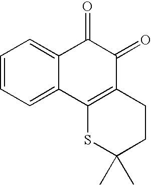 | C₁₅H₁₄O₂S | 258.34 |
| 34 | 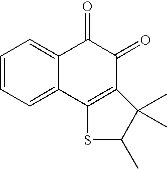 | C₁₅H₁₄O₂S | 258.34 |
| 35 | 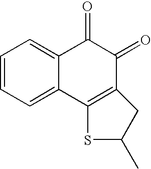 | C₁₃H₁₀O₂S | 230.28 |
| 36 | 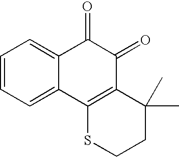 | C₁₅H₁₄O₂S | 258.34 |
| 37 | 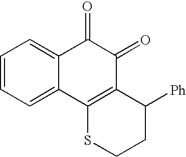 | C₁₉H₁₄O₂S | 306.38 |
| 38 | 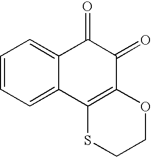 | C₁₂H₈O₃S | 232.26 |
| 39 | 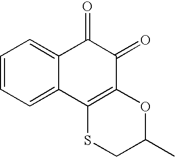 | C₁₃H₁₀O₃S | 246.28 |
| 40 | 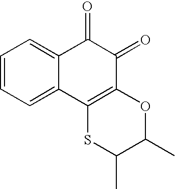 | C₁₄H₁₂O₃S | 260.31 |
| 41 | 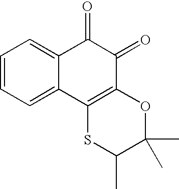 | C₁₅H₁₄O₃S | 274.34 |
| 42 | 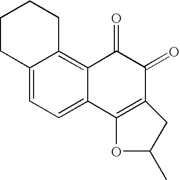 | C₁₇H₁₆O₃ | 268.31 |
| 43 | 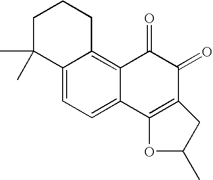 | C₁₉H₂₀O₃ | 296.36 |
| 44 | 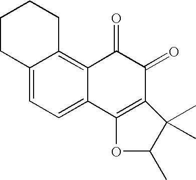 | C₁₉H₂₀O₃ | 296.36 |
| 45 | 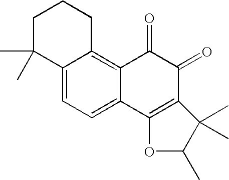 | C₂₁H₂₄O₃ | 324.41 |
| 46 | 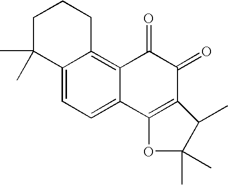 | C₂₁H₂₄O₃ | 324.41 |
| 47 | 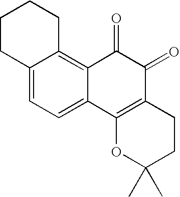 | C₁₉H₂₀O₃ | 296.36 |
| 48 | 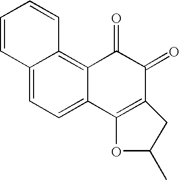 | C₁₇H₁₂O₃ | 264.28 |
| 49 | 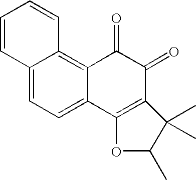 | C₁₉H₁₆O₃ | 292.33 |
| 50 | 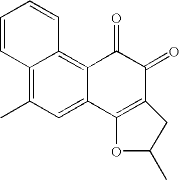 | C₁₈H₁₄O₃ | 278.30 |
| 51 | 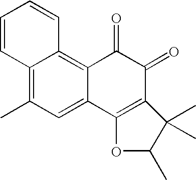 | C₂₀H₁₈O₃ | 306.36 |
| 52 | 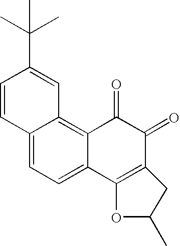 | C₂₁H₂₀O₃ | 320.38 |
| 53 | 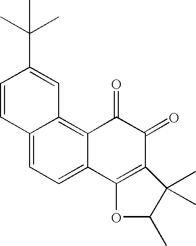 | C₂₃H₂₄O₃ | 348.43 |
| 54 | 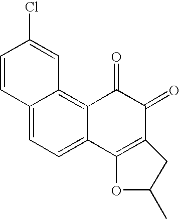 | C₁₇H₁₁ClO₃ | 298.72 |
| 55 | 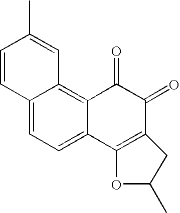 | C₁₈H₁₄O₃ | 278.30 |
| 56 | 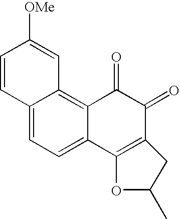 | C₁₈H₁₄O₄ | 294.30 |
| 57 | 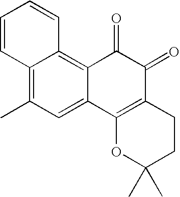 | C₂₀H₁₈O₃ | 306.36 |
| 58 | 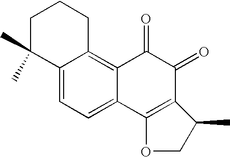 | C₁₈H₁₈O₃ | 282.33 |
| 59 | 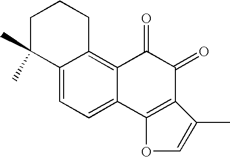 | C₁₈H₁₆O₃ | 280.33 |
| 60 | 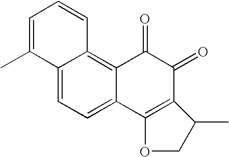 | C₁₈H₁₄O₃ | 278.33 |
| 61 | 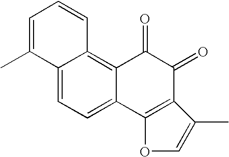 | C₁₈H₁₂O₃ | 276.33 |

The naphthoquinone-based compound is a hydrophobic substance and may be extracted, isolated and purified from crude drugs or otherwise may be synthesized by organic synthesis. When the naphthoquinone-based compound is present in the crystalline state, it exhibits substantially no solubility in water.

On the other hand, micronization of the naphthoquinone-based compound according to the present invention can significantly increase solubility of the drug compound, which consequently results in an improved in vivo absorption rate thereof.

The naphthoquinone-based compound exhibits poor water-solubility, as discussed above. As used herein, the tam “poor water-solubility” means that the compound has a solubility of 10 mg/mL or less, more preferably 1 mg/mL or less in a neutral or acidic aqueous solution.

In the naphthoquinone-based compound according to the present invention, the active ingredient may have a crystalline structure with a high degree of crystallinity, or a crystalline structure with a low degree of crystallinity. Preferably, the active ingredient is composed of the crystalline structure with a low crystallinity degree, which can solve the problems associated with poor solubility of the compound of Formula 1 or 2 and increase the solubility thereof. As used herein, the term “low degree of crystallinity” refers to a crystallinity of 50% or less, and encompasses an amorphous structure from which the intrinsic crystallinity of the material was completely lost.

Preferably, the composition of the present invention may further comprise a desiccant. Incorporation of the desiccant inhibits recrystallization of the naphthoquinone-based compound over time and therefore enables maintenance of increased solubility of a drug compound due to micronization of particles. These facts, as will be illustrated hereinafter, can be clearly confirmed from Example 9 showing that addition of calcium silicate to a composition leads to a significant loss of body weight. Further, the desiccant serves to suppress flocculation and aggregation of the pharmaceutical composition while not adversely affecting therapeutic effects of the naphthoquinone-based compound.

Preferred examples of the desiccant may include, but are not limited to, colloidal silica, light anhydrous silicic acid, heavy anhydrous silicic acid, sodium chloride, calcium silicate, potassium aluminosilicate, calcium aluminosilicate, and the like. These materials may be used alone or in any combination thereof.

Addition of the desiccant may be made at any step of the composition preparation process. For example, the desiccant may be added during the micronization process of the naphthoquinone-based compound or otherwise may be added during the preparation process of the composition.

As used herein, the term “micronized particle” means the particle whose the particle size is 1000 μm or less, and encompasses the nano particle and the micro particle. The term “nano-” is defined as a range of 1 to 1000 nm, and the “micro-” is defined as a range of 1 to 1000 μm. There is no particular limit to the form of particle, preferably, particles with sphere or hollow.

A decreasing particle diameter of the micronized particle in accordance with the present invention leads to an increasing specific surface area, thereby increasing the dissolution rate and solubility. However, an excessively small particle diameter makes it difficult to prepare fine particles having such a size and also brings about agglomeration or aggregation of particles which may result in deterioration of the solubility.

Therefore, the particle size of the micronized particles of naphthoquinone-based compound is desirable when the mean diameter of 90% or more of the particles (X90) is 30 μm or less, more preferably 1 mm to 20 μm and particularly preferably 1 nm to 10 μm. In another embodiment, the particle size of the micronized particles is desirable when the mean diameter of 50% or more of the particles (X50) is 10 μm or less, more preferably 1 mm to 5 μm, and particularly preferably 1 nm to 3 μm.

Further, the micronized particles are desirable when having the particle diameters of a narrow distribution in view of the even solubility. In a desirable example, 90% or more of the micronized particles are within the range of 10% of the mean particle diameter, preferably the range of 5% of the mean particle diameter, particularly the range of 2% of the mean particle diameter,

As used herein, the terms “diameter”, “particle diameter” and “mean diameter” mean the number mean diameter.

As used herein, the micronized naphthoquinone-based compound is intended to include a micronized form of only the compound and a micronized form of the mixture of the compound and additives such as a surfactant.

The present invention presents the activity and therapeutic effects of the drug due to the increased bioavailability thereof, via treatment of diseased animals with the micronized particles of naphthoquinone-based compound.

Preferably, the particle micronization method for preparation of the micronized particles of naphthoquinone-based compound may include milling, precipitation, high-pressure homogenization, and supercritical micronization. These methods may be used alone or in any combination thereof to obtain very finely divided particles.

The milling is a method of grinding the active ingredient into fine particles by applying strong physical force to active ingredient particles. The mechanical milling may be carried out by using a variety of milling processes such as jet milling, ball milling, vibration milling, hammer milling, and the like. Particularly preferred is jet milling which can be carried out using an air pressure, at a temperature of 40° C. or less.

Preferably, a surfactant may be additionally added to prevent the particle agglomeration or aggregation which may occur during preparation of the micronized particles of naphthoquinone-based compound. There is no particular limit to a time point of surfactant addition. For example, the surfactant may be mixed before, during and/or after the micronization process.

Through a series of these processes as described above, the present inventors could easily ground a sparingly-soluble drug, such as naphthoquinone-based compounds, into microparticles which was difficult to achieve only by a conventional milling method. Further, the incorporation of the surfactant resulted in the production of a small-sized, stable and readily-dispersible naphthoquinone-based compound which can be more effectively micronized into a finer particle size, involves no agglomeration and flocculation due to interaction of particles, and does not need a carrier for inclusion or encapsulation of particles. Further, since the thus-prepared micronized particles of the naphthoquinone-based compound contain a tiny amount of the surfactant, such particles are relatively readily dispersible in water to form a stable suspension or emulsion, can exhibit an increased absorption rate, and can be formulated into an oral preparation. Accordingly, upon using the same amount of the naphthoquinone-based compound as the active ingredient, the oral preparation thus formulated can exert relatively superior bioavailability, as compared to other dosage formulations.

In the present invention, the surfactant is a material that physically binds to a surface of the naphthoquinone-based compound, with exclusion of a material that chemically binds to the naphthoquinone-based compound surface, and is a concept encompassing vehicles used for conventional pharmaceutical formulation, including emulsifiers and polymers. Preferably, the surfactant may be an organic or inorganic drug vehicle known in the art, and may include, for example high-molecular weight polymers, low-molecular weight oligomers, and naturally-occurring surfactants.

There is no particular limit to the emulsifiers, for example, lipid such as lecithin, phosphtidylcoline, phosphatidylethanolamine, phosphatidyl-serine, phosphatidylinositol, derivatives thereof; ester derivative of fatty acid such as glyceryl stearate, sorbitan palmitate; sorbitan-based emulsifier, such as Polyoxyethylene sorbitan monolaurate (Tween 20), Polyoxyethylene sorbitan monopalmitate (Tween 40), Polyoxyethylene sorbitan monostearate (Tween 60), Polyoxyethylene sorbitan monooleate (Tween 80), Sorbitan monolaurate (Span 20), Sorbitan monolaurate (Span 20), Sorbitan monostearate (Span 60), Sorbitan monooleate (Span 80), and the like.

The surfactants may includes, but is not limited to, a naturally-occurring surfactants such as casein, gelatin, phospholipids, tragacanth, lecithin, acacia gum, cholesterol, stearic acid, benzalkonium chloride, stearin calcium, glyceryl monostearate, natural phospholipids, waxes, enteric resins, paraffin, acacia; nonionic surfactants such as polyoxyethylene fatty alcohol ethers, sorbitan esters, glycerol monostearate, polyethylene glycols, cetyl alcohol, cetostearyl alcohol, stearyl alcohol, poloxamers, polaxamines, methylcellulose, hydroxycellulose, hydroxy propylcellulose, hydroxy propylmethylcellulose, noncrystalline cellulose, synthetic phospholipids; anionic surfactants such as potassium laurate, triethanolamine stearate, sodium lauryl sulfate, alkyl polyoxyethylene sulfates, sodium alginate, dioctyl sodium sulfosuccinate, negatively charged phosphatidylserine, phosphatidyl glycerol, phosphatidyl inosite, phosphatidylserine, phosphatidic acid, negatively charged glyceryl ester, sodium carboxymethylcellulose, calcium carboxymethylcellulose; and cationic surfactants such as quaternary ammonium compound, benzalkonium chloride, cetyltrimethylammonium bromide, lauryldimethylbenzyl-ammonium chloride, colloidal clays, bentonite, veegum, natural synthetic phospholipids, phosphatidylcholine, phosphatidylethanolamine, phosphatidylserine, phosphatidylinositol, phosphatidylglycerol, phosphatidic acid, lysophospholipid, egg or soybean phospholipids. Preferred examples of the surfactant may include sodium lauryl sulfate, phosphorylated lipid, calcium silicate, chitosan, or sucrose palmitate.

A content of the surfactant may be in a range of preferably 0.1 to 50%, more preferably 0.5 to 30%, and particularly preferably 0.5 to 15%, based on the total weight of the naphthoquinone-based compound and surfactant.

The size of the micronized particles may be adjusted by controlling a grinding time or the surfactant content.

When particle reduction is carried out using the above grinding process, the micronization process may be preferably carried out under refrigeration conditions to reduce particle size growth and particle aggregation. Further, the particle size growth and particle aggregation can be minimized by storage of micronized particles at a low temperature.

Meanwhile, the aqueous solution may be dried by direct drying, or spray coating on vehicles, or spraying on vehicles using a fluid-bed spray coater.

The pharmaceutical composition may further comprise a water-soluble polymer and/or a solubilizer, if necessary.

The water-soluble polymer is of help to prevent aggregation of the particulate active ingredients, by rendering molecules of the compound of Formula 1 or 2 or surroundings of the particles hydrophilic to consequently enhance water solubility, and preferably to maintain the amorphous state of the compound of Formula 1 or 2 as an active ingredient.

Preferably, the water-soluble polymer is a pH-independent polymer, and can bring about crystallinity loss and enhanced hydrophilicity of the active ingredient, even under the between- and within-individual variation of the gastrointestinal pH.

Examples of the water-soluble polymer may include cellulose derivatives, polyvinyl-based polymers, polyglycol-based polymers, polyalkene oxide, polyalkene glycol, methacrylic acid-ethyl acrylate copolymers, corn protein extracts, sodium alginate, alginic acid, agar, carageenan, pectin, guar gum, locust bean gum, xanthan gum, gelan gum, gum arabic, shellac and the like.

An excessive content of the water-soluble polymer which is higher than a given level provides no further increased solubility, but disadvantageously brings about various problems such as overall increases in the hardness of the formulation, and non-penetration of an eluent into the formulation, by formation of films around the formulation due to excessive swelling of water-soluble polymers upon exposure to the eluent. Accordingly, the solubilizer is preferably added to maximize the solubility of the formulation by modifying physical properties of the naphthoquinone-based compound.

In this respect, the solubilizer serves to enhance solubilization and wettability of the sparingly-soluble compound, and can significantly reduce the bioavailability variation of the naphthoquinone-based compound originating from diets and the time difference of drug administration after dietary uptake. The solubilizer may be selected from conventionally widely used surfactants or amphiphiles, and specific examples of the solubilizer may refer to the surfactants as defined above.

The disintegration-promoting agent serves to improve the drug release rate, and enables rapid release of the drug at the target site to thereby increase bioavailability of the drug. Preferred examples of the disintegration-promoting agent may include, but are not limited to, at least one selected from the group consisting of Croscarmellose sodium, Crospovidone, calcium carboxymethylcellulose, starch glycolate sodium and lower substituted hydroxypropyl cellulose. Preferred is Croscarmellose sodium.

Upon taking into consideration various factors as described above, it is preferred to add 10 to 1000 parts by weight of the water-soluble polymer, 1 to 30 parts by weight of the disintegration-promoting agent and 0.1 to 20 parts by weight of the solubilizer, based on 100 parts by weight of the active ingredient.

In addition to the above-mentioned ingredients, other materials known in the art in connection with formulation may be optionally added, if necessary.

Further, the pharmaceutical composition may further comprise a pharmaceutically acceptable carrier, diluent, vehicles or any combination thereof, if necessary. These optional ingredients may also be added at various steps, as illustrated in addition of the desiccant.

A term “carrier” means a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

A term “diluent” defines chemical compounds diluted in water that will dissolve the compound of interest as well as stabilize the biologically active form of the compound. Salts dissolved in buffered solutions are utilized as diluents in the art. One commonly used buffer solution is phosphate buffered saline (PBS) because it mimics the ionic strength conditions of human body fluid. Since buffer salts can control the pH of a solution at low concentrations, a buffer diluent rarely modifies the biological activity of a compound.

Although there is no particular limit to kinds of carriers, the carrier may be preferably one conventionally used in the art depending upon desired formulations, for example at least one selected from the group consisting of solid carriers such as starch, lactose, mannitol, carboxymethylcellulose, corn starch and inorganic salts; liquid carriers such as distilled water, physiological saline, aqueous glucose solutions, alcohols such as ethanol, propylene glycol, and polyethylene glycol; and oily carriers such as various animal and vegetable oils, white Vaseline, paraffin and wax.

Examples of vehicles may include fillers such as lactose, sucrose, mannitol and sorbitol, corn starch, wheat starch, rice starch, potato starch, gelatin, tragacanth gum, methyl cellulose, hydroxypropylmethylcellulose, sodium carboxymethylcellulose, and/or cellulosic materials such as polyvinylpyrrolidone (PVP).

Preferably, the composition of the present invention may be formulated into a composition containing one or more cosmetically or sitologically acceptable carriers or vehicles. That is, the composition may be prepared into a composition in the form of a cosmetic or cosmetic additive, a beverage or beverage additive, a food or food additive, or a functional health food.

As used herein, the term “functional health food” refers to a food in which the composition of the present invention is added to a general food to thereby improve functions thereof. For this purpose, the composition of the present invention may be added to general foods or may be prepared in the form of capsules, powders, suspensions and the like. Intake of such a functional health food containing the composition of the present invention provides beneficial effects for health, and exhibits advantages in that there are no adverse side effects which may occur upon long-term administration of drugs because a food is used as the raw material, unlike conventional drugs.

If it is desired to use the composition of the present invention as a food additive, the composition may be added alone, or otherwise may be used in conjunction with other foods or food ingredients, or may be used appropriately according to any conventional method. A mixed amount of the active ingredient may be suitably determined depending upon the desired uses and applications (prophylactic, health or therapeutic treatment). There is no particular limit to kinds of the above-mentioned foods.

When it is desired to use the composition of the present invention as a cosmetic raw material, the composition can be added by itself or can be used in conjunction with other cosmetic ingredients, or may be used appropriately according to other conventional methods. A mixed amount of the active ingredient may be suitably determined depending upon the purpose of use thereof.

The pharmaceutical composition in accordance with the present invention may be preferably an oral pharmaceutical composition which is prepared into an intestine-targeted formulation.

Generally, an oral pharmaceutical composition passes through the stomach upon oral administration, is largely absorbed by the small intestine and then diffused into all the tissues of the body, thereby exerting therapeutic effects on the target tissues.

In this connection, the oral pharmaceutical composition according to the present invention enhances bioabsorption and bioavailability of compounds of Formula 1 or 2 as an active ingredient via intestine-targeted formulation of the active ingredient. More specifically, when the active ingredient in the pharmaceutical composition according to the present invention is primarily absorbed in the stomach, and upper parts of the small intestine, the active ingredient absorbed into the body directly undergoes liver metabolism which is then accompanied by substantial degradation of the active ingredient, so it is impossible to exert a desired level of therapeutic effects. On the other hand, it is expected that when the active ingredient is largely absorbed around and downstream of the lower small intestine, the absorbed active ingredient migrates via lymph vessels to the target tissues to thereby exert high therapeutic effects.

Further, as it is constructed in such a way that the pharmaceutical composition according to the present invention targets up to the colon which is a final destination of the digestion process, it is possible to increase the in vivo retention time of the drug and it is also possible to minimize decomposition of the drug which may take place due to the body metabolism upon administration of the drug into the body. As a result, it is possible to improve pharmacokinetic properties of the drug, to significantly lower a critical effective dose of the active ingredient necessary for the treatment of the disease, and to obtain desired therapeutic effects even with administration of a trace amount of the active ingredient. Further, in the oral pharmaceutical composition, it is also possible to minimize the absorption variation of the drug by reducing the between- and within-individual variation of the bioavailability which may result from intragastric pH changes and dietary uptake patterns.

Therefore, the intestine-targeted formulation according to the present invention is configured such that the active ingredient is largely absorbed in the small and large intestines, more preferably in the jejunum, and the ileum and colon corresponding to the lower small intestine, particularly preferably in the ileum or colon.

The intestine-targeted formulation may be designed by taking advantage of numerous physiological parameters of the digestive tract, through a variety of methods. In one preferred embodiment of the present invention, the intestine-targeted formulation may be prepared by (1) a formulation method based on a pH-sensitive polymer, (2) a formulation method based on a biodegradable polymer which is decomposable by an intestine-specific bacterial enzyme, (3) a formulation method based on a biodegradable matrix which is decomposable by an intestine-specific bacterial enzyme, or (4) a formulation method which allows release of a drug after a given lag time, and any combination thereof.

Specifically, the intestine-targeted formulation (1) using the pH-sensitive polymer is a drug delivery system which is based on pH changes of the digestive tract. The pH of the stomach is in a range of 1 to 3, whereas the pH of the small and large intestines has a value of 7 or more, which is higher compared to that of the stomach. Based on this fact, the pH-sensitive polymer may be used in order to ensure that the pharmaceutical composition reaches the lower intestinal parts without being affected by pH fluctuations of the digestive tract. Examples of the pH-sensitive polymer may include, but are not limited to, at least one selected from the group consisting of methacrylic acid-ethyl acrylate copolymer (Eudragit: Registered Trademark of Rohm Pharma GmbH), hydroxypropylmethyl cellulose phthalate (HPMCP) and a mixture thereof.

Preferably, the pH-sensitive polymer may be added by a coating process. For example, addition of the polymer may be carried out by mixing the polymer in a solvent to form an aqueous coating suspension, spraying the resulting coating suspension to form a film coating, and drying the film coating.

The intestine-targeted formulation (2) using the biodegradable polymer which is decomposable by the intestine-specific bacterial enzyme is based on the utilization of a degradative ability of a specific enzyme that can be produced by enteric bacteria. Examples of the specific enzyme may include azoreductase, bacterial hydrolase glycosidase, esterase, polysaccharidase, and the like.

When it is desired to design the intestine-targeted formulation using azoreductase as a target, the biodegradable polymer may be a polymer containing an azoaromatic linkage, for example, a copolymer of styrene and hydroxyethylmethacrylate (HEMA). When the polymer is added to the formulation containing the active ingredient, the active ingredient may be liberated into the intestine by reduction of an azo group of the polymer via the action of the azoreductase which is specifically secreted by enteric bacteria, for example, Bacteroides fragilis and Eubacterium limosum.

When it is desired to design the intestine-targeted formulation using glycosidase, esterase, or polysaccharidase as a target, the biodegradable polymer may be a naturally-occurring polysaccharide or a substituted derivative thereof. For example, the biodegradable polymer may be at least one selected from the group consisting of dextran ester, pectin, amylase, ethyl cellulose and a pharmaceutically acceptable salt thereof. When the polymer is added to the active ingredient, the active ingredient may be liberated into the intestine by hydrolysis of the polymer via the action of each enzyme which is specifically secreted by enteric bacteria, for example, Bifidobacteria and Bacteroides spp. These polymers are natural materials, and have an advantage of low risk of in vivo toxicity.

The intestine-targeted formulation (3) using the biodegradable matrix which is decomposable by an intestine-specific bacterial enzyme may be a form in which the biodegradable polymers are cross-linked to each other and are added to the active ingredient or the active ingredient-containing formulation. Examples of the biodegradable polymer may include naturally-occurring polymers such as chondroitin sulfate, guar gum, chitosan, pectin, and the like. The degree of drug release may vary depending upon the degree of cross-linking of the matrix-constituting polymer.

In addition to the naturally-occurring polymers, the biodegradable matrix may be a synthetic hydrogel based on N-substituted acrylamide. For example, there may be used a hydrogel synthesized by cross-linking of N-tert-butylacryl amide with acrylic acid or copolymerization of 2-hydroxyethyl methacrylate and 4-methacryloyloxyazobenzene, as the matrix. The cross-linking may be, for example an azo linkage as mentioned above, and the formulation may be a form where the density of cross-linking is maintained to provide the optimal conditions for intestinal drug delivery and the linkage is degraded to interact with the intestinal mucous membrane when the drug is delivered to the intestine.

Further, the intestine-targeted formulation (4) with time-course release of the drug after a lag time is a drug delivery system utilizing a mechanism that is allowed to release the active ingredient after a predetermined time irrespective of pH changes. In order to achieve enteric release of the active drug, the formulation should be resistant to the gastric pH environment, and should be in a silent phase for 5 to 6 hours corresponding to a time period taken for delivery of the drug from the body to the intestine, prior to release of the active ingredient into the intestine. The time-specific delayed-release formulation may be prepared by addition of the hydrogel prepared from copolymerization of polyethylene oxide with polyurethane.

Specifically, the delayed-release formulation may have a configuration in which the formulation absorbs water and then swells while it stays within the stomach and the upper digestive tract of the small intestine, upon addition of a hydrogel having the above-mentioned composition after applying the drug to an insoluble polymer, and then migrates to the lower part of the small intestine which is the lower digestive tract and liberates the drug, and the lag time of drug is determined depending upon a length of the hydrogel.

As another example of the polymer, ethyl cellulose (EC) may be used in the delayed-release dosage formulation. EC is an insoluble polymer, and may serve as a factor to delay a drug release time, in response to swelling of a swelling medium due to water penetration or changes in the internal pressure of the intestines due to a peristaltic motion. The lag time may be controlled by the thickness of EC. As an additional example, hydroxypropylmethyl cellulose (HPMC) may also be used as a retarding agent that allows drug release after a given period of time by thickness control of the polymer, and may have a lag time of 5 to 10 hours.

Micronized particles of naphthoquinone-based compound as an active component can contain by therapeutically effective amount, wherein, the term “therapeutically effective amount” means an amount of an active ingredient that is effective to relieve or reduce to some extent one or more of the symptoms of the disease in need of treatment, or to retard initiation of clinical markers or symptoms of a disease in need of prevention, when the compound is administered.

Thus, a therapeutically effective amount refers to an amount of the active ingredient which exhibit effects of (i) reversing the rate of progress of a disease; (ii) inhibiting to some extent further progress of the disease; and/or, (iii) relieving to some extent (or, preferably, eliminating) one or more symptoms associated with the disease. The therapeutically effective amount may be empirically determined by experimenting with the compounds concerned in known in vivo and in vitro model systems for a disease in need of treatment.

When the pharmaceutical composition of the present invention is formulated into a unit dosage form, the compound of Formula I as the active ingredient is preferably contained in a unit dose of about 0.1 to 5,000 mg. The amount of the compound administered will be determined by the attending physician, depending upon body weight and age of patients being treated, characteristic nature and the severity of diseases. However, for adult patients, a dose of the active ingredient administered to the patient is typically within a range of about 1 to 1000 mg/kg per day, depending upon frequency and intensity of administration. For intramuscular or intravenous administration into adult patients, the total amount of about 1 to 500 mg per day as a single dose will be sufficient, preferably, it might be designed that micronized particles of naphthoquinone-based compound will be administered at least over 10 mg per day.

The pharmaceutical compositions in accordance with the present invention are effective for prevention and treatment of metabolic diseases, Restenosis, Impotence, prostatic disease, hypertension, cardiac diseases, renal diseases and glaucoma, degenerative diseases, and mitochondrial dysfunction-related diseases, wherein the metabolic diseases may include, but are not limited to, obesity, an obesity complication, a liver disease, arteriosclerosis, cerebral apoplexy, myocardial infarction, a cardiovascular disease, an ischemic disease, diabetes, a diabetes-related complication or an inflammatory disease.

Some contents regarding the naphthoquinone-based compounds of the present invention and therapeutic effects thereof are disclosed in Korean Patent Application Nos. 2004-0116339, 2006-0014541, 2007-0136105, 2007-0139740, 2007-0141303, PCT/KR2007/006012, PCT/KR2007/006013 and PCT/KR2007/006011 assigned to the present applicant, the disclosures of which are incorporated by reference herein in their entirety.

### BRIEF DESCRIPTION OF THE DRAWINGS

The above and other objects, features and other advantages of the present invention will be more clearly understood from the following detailed description taken in conjunction with the accompanying drawings, in which:

FIG. 1 depicts particle size distribution of micronized cryptotanshinone using supercritical fluid;

FIG. 2 depicts particle size distribution of micronized cryptotanshinone prior to use of supercritical fluid;

FIG. 3 is a graph showing changes of the dissolution rate of micronized particles of Compound 1 with respect to the passage of time, according to Example 15;

FIG. 4 is a graph showing changes of the dissolution rate of micronized particles of Compound 58 extracts with respect to the passage of time, according to Example 15;

FIG. 5 is a graph showing changes of the dissolution rate of micronized particles of Compound 5 with respect to the passage of time, according to Example 15;

FIG. 6 is a graph showing changes of the dissolution rate of micronized particles of Compounds 44 and 51 with respect to the passage of time, according to Example 15;

FIG. 7 is a graph showing changes of the dissolution rate of micronized particles of compound 1 and granulated particles with respect to the passage of time, according to Example 16.

### DETAILED DESCRIPTION OF PREFERRED EMBODIMENTS

Now, the present invention will be described in more detail with reference to the following Examples. These examples are provided only for illustrating the present invention and should not be construed as limiting the scope and spirit of the present invention.

### Example 1

### Preparation of Micronized Particles of Compound 58 (Cryptotanshinone) Using Supercritical Micronization

In order to develop a supercritical micronization process, 5 g of cryptotanshinone having a particle size distribution of 1 to 100 μm and an average particle size of 30 μm, and 0.5 g of sodium lauryl sulfate were dissolved in 45 mL of methylene chloride. Thereafter, the resulting solution of cryptotanshinone in methylene chloride was injected at a flow rate of 1 to 2 mL/min via a nozzle orifice with a diameter of 1/16 inch into a reaction vessel which was set to a temperature of 30° C., 80 bar, and 2,000 rpm and was filled with liquefied carbon dioxide, whereas carbon dioxide was injected at a flow rate of 10 to 20 mL/min via a nozzle orifice with a diameter of 1/16 inch using an ISCO pump.

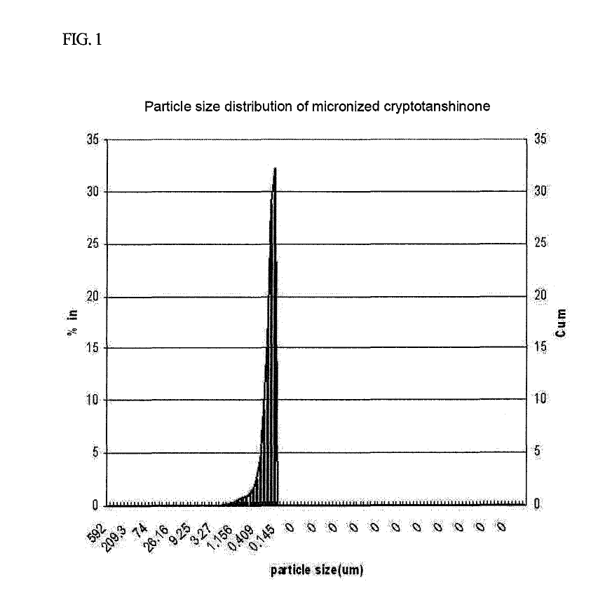

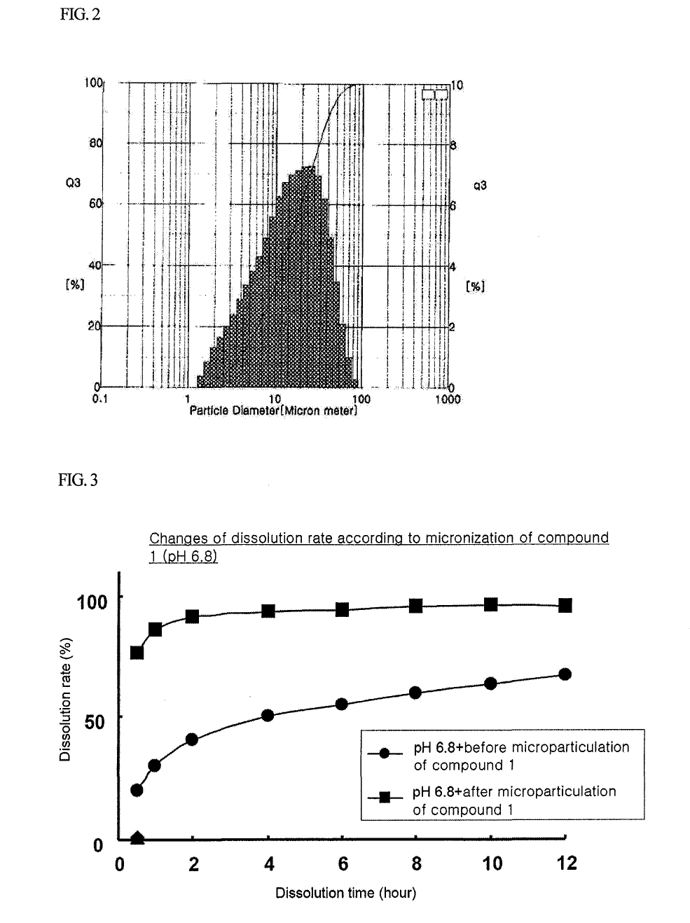

In order to recover precipitates of small-sized particles from the liquid carbon dioxide solution as an insoluble solvent in the reaction vessel, a volume of carbon dioxide which is 15-fold that of the reaction vessel was flowed to remove the remaining organic solvent and the pressure of the reaction vessel was reduced to recover micronized particles of cryptotanshinone. A size of micronized particles of cryptotanshinone using supercritical fluid and a size of micronized particles prior to application of supercritical micronization are shown in FIGS. 1 and 2, respectively. As shown in FIGS. 1 and 2, more than 95% of particles are comprised of particles having a volume diameter of 0.1 to 2 μm.

### Example 2

### Preparation of Micronized Particles of Compound 58 (Cryptotanshinone) Using Jet Mill

Micronizing of cryptotanshinone was carried out using a Jet mill (SJ-100, Nisshin, Japan). Operation was run at a supply pressure of 0.65 Mpa, and a feed rate of 50 to 100 g/hr. 0.2 g of sodium lauryl sulfate (SLS) and 10 g of cryptotanshinone were mixed and ground. Micronized particles were recovered and a particle size was determined in terms of particle diameter by zeta potential measurement. An average particle size was 1500 nm.

### Example 3

### Preparation of Micronized Particles of Compound 58 (Cryptotanshinone) Using High-Pressure Homogenizer

15 g of cryptotanshinone, which was mixed with 0.2% (w/w) of sodium lauryl sulfate, was dispersed in 600 mL of an aqueous solution, and then processed in an Ulta-Turrax T25 Basic homogenizer (IKA-Werke GmbH, Germany) at 24,000 rpm for 10 min to narrow a particle size distribution. First, a size of particles was made uniform by one or two passes of low-pressure homogenization through a microfluidizer processor (M-110EH, Microfluidics Corporation) at 15° C. and 7,000 psi, followed by three passes of high-pressure homogenization at 15,000 psi. The temperature of the apparatus was maintained at 15° C. using heat exchange equipment in all the operations. The cryptotanshinone samples were recovered and then a particle size was assayed. Some of the samples were used for efficacy evaluation. More than 95% of particles passed through the microfluidizer as described above were comprised of particles having a volume diameter of 0.1 to 2 μm.

### Example 4

### Preparation of Micronized Particles of Compound 60 (15,16-dihydrotanshinone) Using High-Pressure Homogenizer

15 g of 15,16-dihydrotanshinone, which was mixed with 0.2% (w/w) of sodium lauryl sulfate, was dispersed in 600 mL of an aqueous solution, and then processed in an Ultra-Turrax T25 Basic homogenizer (IKA-Werke GmbH, Germany) at 24,000 rpm for 10 min to narrow a particle size distribution. First, a size of particles was made uniform by one or two passes of low-pressure homogenization through a microfluidizer processor (M-110EH, Microfluidics Corporation) at 15° C. and 7,000 psi, followed by three passes of high-pressure homogenization at 15,000 psi. The temperature of the apparatus was maintained at 15° C. using heat exchange equipment in all the operations. The 15,16-dihydrotanshinone samples were recovered and then a particle size was assayed. Some of the samples were used for efficacy evaluation. More than 95% of particles passed through the microfluidizer as described above were comprised of particles having a volume diameter of 0.1 to 2 μm.

### Example 5

### Preparation of Micronized Particles of Compound 59 (Tanshinone IIa) Using High-Pressure Homogenizer

15 g of tanshinone IIA, which was mixed with 0.2% (w/w) of sodium lauryl sulfate, was dispersed in 600 mL of an aqueous solution, and then processed in an Ultra-Turrax T25 Basic homogenizer (IKA-Werke GmbH, Germany) at 24,000 rpm for 10 min to narrow a particle size distribution. First, a size of particles was made uniform by five passes of low-pressure homogenization through a microfluidizer processor (M-110EH, Microfluidics Corporation) at 15° C. and 7,000 psi, followed by 50 passes of high-pressure homogenization at 24,000 psi. The temperature of the apparatus was maintained at 15° C. using heat exchange equipment in all the operations. The tanshinone IIA samples were recovered and then a particle size was assayed. Some of the samples were used for efficacy evaluation. More than 95% of particles passed through the microfluidizer as described above were comprised of particles having a volume diameter of 0.1 to 2 μm.

### Example 6

### Effects of Micronized Particles of Compound 58 (Cryptotanshinone) on Animals

1 g of a simply finely-divided powder of cryptotanshinone, and 1 g of each micronized particles of cryptotanshinone compound according to the procedures of Examples 1 to 3 were mixed with 10 mL of distilled water, and the resulting mixtures were processed in a sonicator for 30 min to prepare suspensions of the cryptotanshinone compounds. 400 mg/kg of these suspensions in terms of cryptotanshinone content was administered to ob/ob mice once a day and changes in the body weight (BW) of animals were examined.

For this purpose, 10-week-old ob/ob male mice (Jackson Lab) as an obese mouse model of type 2 diabetes were purchased from Orient Co., Ltd., Korea and were allowed to acclimate to a new environment of the breeding room for 10 days prior to experiments. Animals were fed a solid feed (P5053, Labdiet) as a laboratory animal chow. The ob/ob male mice were housed and allowed to acclimate to a new environment for 10 days, in a breeding room maintained at a temperature of 22±2° C., humidity of 55±5%, and a 12-h light/dark (L/D) cycle (light from 8:00 a.m. to 8:00 p.m.). According to a randomized blocks design, the thus-acclimated animals were randomly divided into five groups, each consisting of 7 animals: a control group with administration of sodium lauryl sulfate (10 mg/kg), a group with administration of a simply finely-divided powder of cryptotanshinone (400 mg/kg), a group with administration of cryptotanshinone subjected to supercritical micronization (Example 1), a group with administration of jet-milled cryptotanshinone (Example 2), and a group with administration of microfluidizer-milled cryptotanshinone (Example 3). Each group of animals was given perorally (PO) 400 mg/kg of drug samples. Animals were fed solid feed pellets and water ad libitum. The results for changes in the body weight of animals are given in Table 1 below. As can be seen from Table 1, it was confirmed that each administration of the micronized particles of cryptotanshinone exhibited a significant loss of body weight at the same dose of the compound, irrespective of micronization methods and equipment, as compared to the group to which the simply finely-divided cryptotanshinone powder was administered.

TABLE 2

| | Amount | | | |
|---|---|---|---|---|
| Drug samples | (mg/kg) | Initial BW (g) | Final BW (g) | BW loss (%) |
| Control | — | 55.9 ± 1.2 | 58.5 ± 2.0 | 4.7 ± 2.1 ↑ |
| Simply finely- | 400 | 55.8 ± 0.5 | 57.5 ± 1.3 | 3.0 ± 0.9 ↑ |
| divided | | | | |
| cryptotanshinone | | | | |
| Example 1 | 400 | 55.8 ± 0.5 | 53.2 ± 1.3 | 4.7 ± 0.9 ↓ |
| Example 2 | 400 | 55.1 ± 0.3 | 52.1 ± 0.5 | 5.4 ± 0.5 ↓ |
| Example 3 | 400 | 57.3 ± 3.5 | 49.7 ± 3.3 | 13.3 ± 2.3↓ |

### Example 7

### Determination of Therapeutically Effective Amount of Drug with Varying Dosages of Micronized Particles of Cryptotanshinone

Each of formulations composed of a mixture of Jet mill-micronized cryptotanshinone at 25 mg/kg, 50 mg/kg and 100 mg/kg with 0.2% (w/w) sodium lauryl sulfate was administered daily for 28 days to DIO (diet-induced obesity) SD rats in which obesity was caused by high-fat diet for 4 weeks. As a control, a surfactant SLS at a concentration of 0.2% was suspended in distilled water and a suspension was then administered to animals. As a result, as can be seen in Table 3 below, a significant loss of body weight was confirmed as compared to the control group, and body weight loss was concentration-dependent.

TABLE 3

| | Amount (mg/kg) | Initial BW (g) | Final BW (g) | BW loss (%) |
|---|---|---|---|---|
| Normal diet | — | 456.3 ± 36.9 | 542.1 ± 46.9 | 18.8 ± 2.6 |
| High-fat diet | — | 506.0 ± 35.2 | 619.5 ± 46.5 | 22.4 ± 2.0 |
| High-fat diet + | 25 | 503.9 ± 35.7 | 589.0 ± 50.4 | 16.9 ± 3.8 |
| cryptotanshinone (25 mg/kg) | | | | |
| High-fat diet + | 50 | 501.6 ± 34.8 | 578.8 ± 54.6 | 15.4 ± 3.7 |
| cryptotanshinone (50 mg/kg) | | | | |
| High-fat diet + | 100 | 498.8 ± 33.9 | 529.8 ± 59.1 | 6.2 ± 5.5 |
| Cryptotanshinone (100 mg/kg) | | | | |

### Example 8

### Effects of Phospholipid on Enhanced Efficacy of Micronized Particles of Cryptotanshinone

In order to ascertain whether phosphatidylcholine (PC) which is a phospholipid maintains a particle size of micronized particles of cryptotanshinone, prevents the particle agglomeration or aggregation, and enhances therapeutic efficacy of cryptotanshinone, an animal experiment was carried out as follows.

For this purpose, 5-week-old C57BL/6 male mice were purchased from Orient Co., Ltd., Korea and were allowed to acclimate to a new environment of the breeding room for 10 days prior to experiments. A DIO (diet-induced obesity) mouse model was established by supplying to animals high-fat diet for 8 weeks. 13-week-old male mice weighing 40 g were used in experiments. Animals were fed a high-fat feed (45 kcal % fat) (D12451, Research Diets Inc., New Brunswick, N.J.) as a laboratory animal chow. The DIO mice were housed and allowed to acclimate to a new environment for 10 days, in a breeding room maintained at a temperature of 22±2° C., humidity of 55±5%, and a 12-h light/dark (L/D) cycle (light from 8:00 a.m. to 8:00 p.m.). According to a randomized blocks design, the thus-acclimated animals were randomly divided into four groups, each consisting of 7 animals: a control group with no administration of any drug sample, a group with administration of a vehicle (PC) only, a group with administration of cryptotanshinone (150 mg/kg), and a group with administration of cryptotanshinone (150 mg/kg) plus PC. Each group of animals was given perorally (PO) drug samples for 40 days. Animals had ad libitum access solid feed pellets and water.

TABLE 4

| | Amount | Initial | | |
|---|---|---|---|---|
| Drug samples | (mg/kg) | BW (g) | Final BW (g) | BW loss (%) |
| Control | — | 44.2 ± 2.1 | 48.8 ± 2.3 | 10.4 ± 2.2 ↑ |
| Vehicle (PC) | 300 | 42.2 ± 2.0 | 46.3 ± 1.5 | 9.7 ± 1.8 ↑ |
| Cryptotanshinone | 150 | 43.2 ± 2.5 | 40.2 ± 1.3 | 6.9 ± 0.9 ↓ |
| alone | | | | |
| Cryptotanshinone + | 150 | 42.7 ± 2.1 | 39.0 ± 2.5 | 8.7 ± 0.8 ↓ |
| PC | | | | |

As can be seen from Table 4, it was confirmed that the combined administration group of cryptotanshinone and phosphatidylcholine (PC) exhibited a significant loss of body weight, as compared to the control group, the vehicle-administered group and the cryptotanshinone-administered group.

### Example 9

### Effects of Calcium Silicate on Enhanced Efficacy of Micronized Particles of Cryptotanshinone

In order to ascertain whether calcium silicate maintains a particle size of micronized particles of cryptotanshinone, prevents the particle agglomeration or aggregation, and enhances therapeutic efficacy of cryptotanshinone, an animal experiment was carried out as follows.

10-week-old ob/ob male mice (Jackson Lab) as an obese mouse model of type 2 diabetes were purchased from Orient Co., Ltd., Korea and were allowed to acclimate to a new environment of the breeding room for 10 days prior to experiments. Animals were fed a solid feed (P5053, Labdiet) as a laboratory animal chow. The ob/ob male mice were housed and allowed to acclimate to a new environment for 10 days, in a breeding room maintained at a temperature of 22±2° C., humidity of 55±5%, and a 12-h light/dark (L/D) cycle (light from 8:00 am. to 8:00 p.m.). According to a randomized blocks design, the thus-acclimated animals were randomly divided into four groups, each consisting of 7 animals: a control group with no administration of any drug sample, a group with administration of a vehicle (calcium silicate) only, a group with administration of cryptotanshinone (400 mg/kg), and a group with administration of cryptotanshinone (400 mg/kg) plus calcium silicate. Each group of animals was given drug samples in admixture with the feed for 20 days. All animals were allowed ad libitum access to water and standard laboratory chow throughout the study period.

The results for changes in the body weight of animals are given in Table 5 below. As can be seen from Table 5, it was confirmed that the combined administration group of cryptotanshinone and calcium silicate exhibited a significant loss of body weight, as compared to the control group, the vehicle-administered group and the cryptotanshinone-administered group.

TABLE 5

| | Amount | | Final | |
|---|---|---|---|---|
| Drug samples | (mg/kg) | Initial BW (g) | BW (g) | BW loss (%) |
| Control | — | 53.2 ± 3.0 | 57.3 ± 2.5 | 7.7 ± 2.3 ↑ |
| Vehicle | 400 | 53.0 ± 1.8 | 57.9 ± 2.0 | 9.2 ± 1.0 ↑ |
| (calcium silicate) | | | | |
| Cryptotanshinone | 400 | 55.8 ± 0.5 | 53.2 ± 1.3 | 4.7 ± 0.9 ↓ |
| alone | | | | |
| Cryptotanshinone + | 400 | 52.1 ± 3.6 | 47.3 ± 2.9 | 9.2 ± 0.5 ↓ |
| calcium silicate | | | | |

### Example 10

### Effects of Chitosan on Enhanced Efficacy of Micronized Particles of Cryptotanshinone

In order to ascertain whether chitosan maintains a particle size of micronized particles of cryptotanshinone, prevents the particle agglomeration or aggregation, and enhances therapeutic efficacy of cryptotanshinone, an animal experiment was carried out as follows.

10-week-old ob/ob male mice (Jackson Lab) as an obese mouse model of type 2 diabetes were purchased from Orient Co., Ltd., Korea and were allowed to acclimate to a new environment of the breeding room for 10 days prior to experiments. Animals were fed a solid feed (P5053, Labdiet) as a laboratory animal chow. The ob/ob male mice were housed and allowed to acclimate to a new environment for 10 days, in a breeding mom maintained at a temperature of 22±2° C., humidity of 55±5%, and a 12-h light/dark (L/D) cycle (light from 8:00 am. to 8:00 p.m.). According to a randomized blocks design, the thus-acclimated animals were randomly divided into four groups, each consisting of 7 animals: a control group with no administration of any drug sample, a group with administration of a vehicle (chitosan) only, a group with administration of cryptotanshinone (400 mg/kg), and a group with administration of cryptotanshinone (400 mg/kg) plus chitosan. Each group of animals was given drug samples in admixture with the feed for 20 days. All animals were allowed ad libitum access to water and standard laboratory chow throughout the study period.

The results for changes in the body weight of animals are given in Table 6 below. As can be seen from Table 6, it was confirmed that the combined administration group of cryptotanshinone and chitosan exhibited a significant loss of body weight, as compared to the control group, the vehicle-administered group and the cryptotanshinone-administered group.

TABLE 6

| | Amount | | Final | |
|---|---|---|---|---|
| Drug samples | (mg/kg) | Initial BW (g) | BW (g) | BW loss (%) |
| Control | — | 53.2 ± 3.0 | 57.3 ± 2.5 | 7.7 ± 2.3 ↑ |
| Vehicle (chitosan) | 800 | 54.0 ± 1.6 | 56.7 ± 2.0 | 5.0 ± 1.0 ↑ |
| Cryptotanshinone | 400 | 55.8 ± 0.5 | 53.2 ± 1.3 | 4.7 ± 0.9 ↓ |
| alone | | | | |
| Cryptotanshinone + | 400 | 55.2 ± 2.2 | 51.3 ± 2.9 | 7.1 ± 1.0 ↓ |
| chitosan | | | | |

### Example 11

### Effects of Sucrose Palmitate on Enhanced Efficacy of Micronized Particles of Cryptotanshinone

In order to ascertain whether sucrose palmitate which is an emulsifier maintains a particle size of micronized particles of cryptotanshinone, prevents the particle agglomeration or aggregation, and enhances therapeutic efficacy of cryptotanshinone, an animal experiment was carried out as follows.

10-week-old ob/ob male mice (Jackson Lab) as an obese mouse model of type 2 diabetes were purchased from Orient Co., Ltd., Korea and were allowed to acclimate to a new environment of the breeding room for 10 days prior to experiments. Animals were fed a solid feed (P5053, Labdiet) as a laboratory animal chow. The ob/ob male mice were housed and allowed to acclimate to a new environment for 10 days, in a breeding room maintained at a temperature of 22±2° C., humidity of 55±5%, and a 12-h light/dark (L/D) cycle (light from 8:00 am. to 8:00 p.m.). According to a randomized blocks design, the thus-acclimated animals were randomly divided into four groups, each consisting of 7 animals: a control group with no administration of any drug sample, a group with administration of a vehicle (sucrose palmitate) only, a group with administration of cryptotanshinone (400 mg/kg), and a group with administration of cryptotanshinone (400 mg/kg) plus sucrose palmitate. Each group of animals was given drug samples in admixture with the feed for 20 days. All animals were allowed ad libitum access to water and standard laboratory chow throughout the study period.

The results for changes in the body weight of animals are given in Table 7 below. As can be seen from Table 7, it was confirmed that the combined administration group of cryptotanshinone and sucrose palmitate exhibited a significant loss of body weight, as compared to the control group, the vehicle-administered group and the cryptotanshinone-administered group.

TABLE 7

| | Disease | Amount | | | |
|---|---|---|---|---|---|
| Drug samples | model | (mg/kg) | Initial BW (g) | Final BW (g) | BW loss (%) |
| Control | ob mice | — | 53.2 ± 3.0 | 57.3 ± 2.5 | 7.7 ± 2.3 ↑ |
| Vehicle (sucrose | ob mice | 800 | 53.0 ± 1.8 | 57.9 ± 2.0 | 9.2 ± 1.0 ↑ |
| palmitate) | | | | | |
| Cryptotanshinone | ob mice | 400 | 55.8 ± 0.5 | 53.2 ± 1.3 | 4.7 ± 0.9 ↓ |
| alone | | | | | |
| Cryptotanshinone + | ob mice | 400 | 55.5 ± 2.2 | 49.5 ± 3.1 | 10.8 ± 0.9 ↓ |
| sucrose | | | | | |
| palmitate | | | | | |

### Example 12

### Effects of Micronized Particles of 15,16-dihydrotanshinone on Animals

In order to confirm therapeutic efficacy of micronized particles of 15,16-dihydrotanshinone, an animal experiment was carried out as follows.

For this purpose, 10-week-old ob/ob male mice (Jackson Lab) as an obese mouse model of type 2 diabetes were purchased from Orient Co., Ltd., Korea and were allowed to acclimate to a new environment of the breeding room for 10 days prior to experiments. Animals were fed a solid feed (P5053, Labdiet) as a laboratory animal chow. The ob/ob male mice were housed and allowed to acclimate to a new environment for 10 days, in a breeding room maintained at a temperature of 22±2° C., humidity of 55±5%, and a 12-h light/dark (L/D) cycle (light from 8:00 am. to 8:00 p.m.). According to a randomized blocks design, the thus-acclimated animals were randomly divided into four groups, each consisting of 7 animals: a control group with no administration of any drug sample, a group with administration of a vehicle (sodium lauryl sulfate) only, a group with administration of 15,16-dihydrotanshinone (before microparticulation, 400 mg/kg) plus sodium lauryl sulfate, and a group with administration of micronized particles of 15,16-dihydrotanshinone (400 mg/kg) plus sodium lauryl sulfate. Each group of animals was given drug samples in admixture with the feed for 20 days. All animals were allowed ad libitum access to water and standard laboratory chow throughout the study period.

The results for changes in the body weight of animals are given in Table 8 below. As can be seen from Table 8, it was confirmed that the combined administration group of micronized particles of 15,16-dihydrotanshinone and sodium lauryl sulfate exhibited a significant loss of body weight, as compared to the control group, the vehicle-administered group and the non-micronized particles of 15,16-dihydrotanshinone-administered group.

TABLE 8

| | Amount | | Final | |
|---|---|---|---|---|
| Drug samples | (mg/kg) | Initial BW (g) | BW (g) | BW loss (%) |
| Control | — | 57.0 ± 1.2 | 59.9 ± 1.8 | 5.1 ± 1.0 ↑ |
| Vehicle (SLS) | 10 | 55.7 ± 2.2 | 57.2 ± 1.8 | 2.7 ± 0.4 ↑ |
| 15,16- | 400 | 57.8 ± 1.3 | 55.4 ± 3.1 | 4.2 ± 0.7 ↓ |
| dihydrotanshinone | | | | |
| before | | | | |
| microparticulation | | | | |
| 15,16- | 400 | 58.0 ± 3.5 | 53.7 ± 3.3 | 7.4 ± 0.8 ↓ |
| dihydrotanshinone | | | | |
| after | | | | |
| microparticulation | | | | |

### Example 13

### Effects of Micronized Particles of Tanshinone IIa on Animals

In order to confirm therapeutic efficacy of micronized particles of tanshinone IIA, an animal experiment was carried out as follows.

For this purpose, 10-week-old ob/ob male mice (Jackson Lab) as an obese mouse model of type 2 diabetes were purchased from Orient Co., Ltd., Korea and were allowed to acclimate to a new environment of the breeding room for 10 days prior to experiments. Animals were fed a solid feed (P5053, Labdiet) as a laboratory animal chow. The ob/ob male mice were housed and allowed to acclimate to a new environment for 10 days, in a breeding room maintained at a temperature of 22±2° C., humidity of 55±5%, and a 12-h light/dark (L/D) cycle (light from 8:00 am. to 8:00 p.m.). According to a randomized blocks design, the thus-acclimated animals were randomly divided into four groups, each consisting of 7 animals: a control group with no administration of any drug sample, a group with administration of a vehicle (sodium lauryl sulfate) only, a group with administration of tanshinone IIA (before microparticulation, 400 mg/kg) plus sodium lauryl sulfate, and a group with administration of micronized particles of tanshinone IIA (400 mg/kg) plus sodium lauryl sulfate. Each group of animals was given drug samples in admixture with the feed for 20 days. All animals were allowed ad libitum access to water and standard laboratory chow throughout the study period.

The results for changes in the body weight of animals are given in Table 9 below. As can be seen from Table 9, it was confirmed that the combined administration group of micronized particles of tanshinone IIA and sodium lauryl sulfate exhibited a significant loss of body weight, as compared to the control group, the vehicle-administered group and the non-micronized particles of tanshinone IIA-administered group.

TABLE 9

| | Amount | | | |
|---|---|---|---|---|
| Drug samples | (mg/kg) | Initial BW (g) | Final BW (g) | BW loss (%) |
| Control | — | 59.5 ± 1.5 | 63.3 ± 1.2 | 6.4 ± 0.2 ↑ |
| Vehicle (SLS) | 10 | 56.2 ± 3.2 | 57.6 ± 1.3 | 2.5 ± 0.5 ↑ |
| Tanshinone IIA | 400 | 60.6 ± 1.5 | 58.4 ± 3.3 | 3.6 ± 1.3 ↑ |
| before | | | | |
| microparticulation | | | | |
| Tanshinone IIA | 400 | 59.3 ± 3.5 | 55.9 ± 3.3 | 5.7 ± 0.4 ↓ |
| after | | | | |
| microparticulation | | | | |

### Example 14

### Effects of Intestine-Targeted Formulation of Micronized Particles of Compound 1 (β-Lapachone) on Animals

In order to confirm therapeutic efficacy of β-lapachone {7,8-dihydro-2,2-dimethyl-2H-naphtho(2,3-b)dihydropyran-7,8-dione} when micronized particles of β-lapachone was prepared into a formulation of an intestine-targeted drug delivery system, an animal experiment was carried out as follows.

0.2 g of sodium lauryl sulfate (SLS) and 10 g of β-lapachone were mixed and ground. To the resulting mixture were added approximately an equal amount of a water-soluble polymer (hydroxypropyl methylcellulose) relative to β-lapachone, and vehicles such as Croscarmellose sodium and light anhydrous silicic acid, followed by spray drying. Then, the spray-dried product was added to an ethanol solution containing about 20% by weight of Eudragit S-100 as a pH-sensitive polymer and about 2% by weight of PEG #6,000 as a plasticizer, and the mixture was then spray-dried to prepare intestine-targeted formulations.

In order to confirm the efficacy of each formulation, 200 mg/kg of the formulation in terms of β-lapachone content was administered to ob/ob mice once a day and changes in the body weight (BW) of animals were examined.

For this purpose, 10-week-old ob/ob male mice (Jackson Lab) as an obese mouse model of type 2 diabetes were purchased from Orient Co., Ltd., Korea and were allowed to acclimate to a new environment of the breeding room for 10 days prior to experiments. Animals were fed a solid feed (P5053, Labdiet) as a laboratory animal chow. The ob/ob male mice were housed and allowed to acclimate to a new environment for 10 days, in a breeding room maintained at a temperature of 22±2° C., humidity of 55±5%, and a 12-h light/dark (L/D) cycle (light from 8:00 am. to 8:00 p.m.). According to a randomized blocks design, the thus-acclimated animals were randomly divided into four groups, each consisting of 7 animals: a control group with administration of sodium lauryl sulfate (10 mg/kg), a group with administration of simply finely-divided powder of β-lapachone (200 mg/kg), a group with administration of jet-milled β-lapachone, and a group with administration of the intestine-targeted formulation of β-lapachone subjected to a milling process. Each group of animals was given perorally (PO) 200 mg/kg of drug samples. Animals were fed solid feed pellets and water ad libitum. The results for changes in the body weight of animals are given in Table 10 below.

TABLE 10

| | Amount | Initial | Final | |
|---|---|---|---|---|
| Drug samples | (mg/kg) | BW (g) | BW (g) | BW loss (%) |
| Control | — | 55.9 ± 1.2 | 58.5 ± 2.0 | 4.7 ± 2.1 ↑ |
| Simply finely- | 200 | 55.8 ± 0.5 | 57.5 ± 1.3 | 3.0 ± 0.9 ↑ |
| divided powder | | | | |
| Jet-milled compound | 200 | 55.8 ± 0.5 | 53.2 ± 1.3 | 4.7 ± 0.9 ↓ |
| Intestine-targeted | 200 | 57.3 ± 3.5 | 49.7 ± 3.3 | 13.3 ± 2.3 ↓ |
| formulation | | | | |

As can be seen from Table 10, the group with administration of the intestine-targeted formulation exhibited the highest decrease (%) of body weight, thus representing that excellent bioavailability is obtained.

### Example 15

### Dissolution Test

Dissolution test was commonly carried out under the following condition.

The dissolution test was carried out according to the paddle method of Korean Phramacopeia. The paddle rate was 75 rpm, the temperature of an effluent was 37° C., and a test solution was a mixture solution of pH 6.8, 1.5% Tween 80. First, the test solution was filtered through a 35 μm Membrane filter and centrifuged. Supernatant fluid was taken, and the content of drug was analyzed by HPLC, and the amount of released drug was measured at different time points. The drugs used for the dissolution test are Compounds 1, 5, 44 and 51 provided in Table 1, and the extract set forth in the Korean Patent No. 10-818586 which contains Compound 58 over 60 wt % (‘Compound 58 Extract’). Microparticulation was carried out using Jet mill or ball mill without any surfactant. The particle size of compounds was determined by HELOS measurement.

Measurement of the particle size was carried out by putting approximately 3 ml of a sample into a cell for particle size measurement and measuring the particle size during 60 seconds per sample. The particle sizes before and after micronization are provided below.

TABLE 11

| | | | Compound | Compound | Compound |
|---|---|---|---|---|---|
| | Compound 1 | Compound 5 | 58 Extract | 44 | 51 |
| Before micronization | X50 = 27.83 | X50 = 3.61 | X50 = 13.39 | X50 = 3.15 | X50 = 5.08 |
| (μm) | X90 = 33.74 | X90 = 20.09 | X90 = 31.32 | X90 = 22.41 | X90 = 21.97 |
| After micronization | X50 = 2.39 | X50 = 2.48 | X50 = 2.10 | X50 = 2.41 | X50 = 3.05 |
| (μm) | X90 = 5.03 | X90 = 5.38 | X90 = 5.23 | X90 = 24.57 | X90 = 13.48 |

The dissolution rates before and after micronization of each compound are provided in Table 12 below. As seen in Table 12, it was ascertained that all dissolution rates as well as the dissolution degree increase after micronization.

TABLE 12

| Dissolution | before | | |
|---|---|---|---|
| rate of 12 | microparticulation | after | Increasing |
| hours | (%) | microparticulation (%) | rate |
| Compound 1 | 67.19 | 95.52 | 42% |
| Compound 5 | 78.12 | 86.12 | 10% |
| Compound 58 | 25.97 | 67.99 | 161% |
| Extract | | | |
| Compound 44 | 13.81 | 41.05 | 191% |
| Compound 51 | 24.08 | 51.3 | 213% |

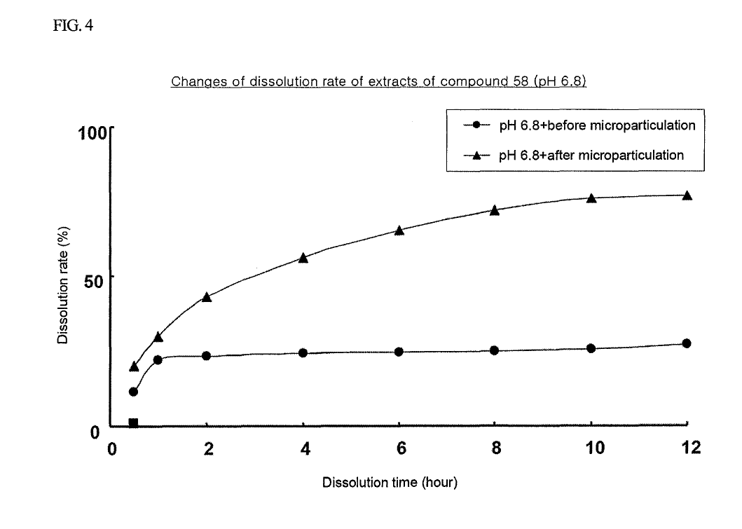

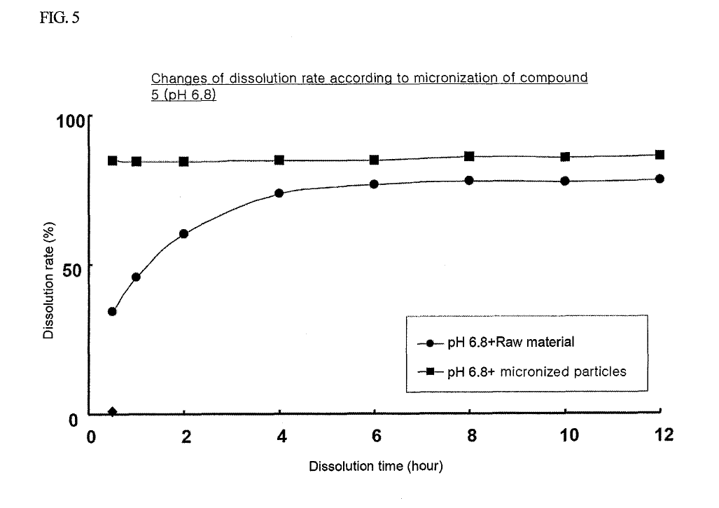

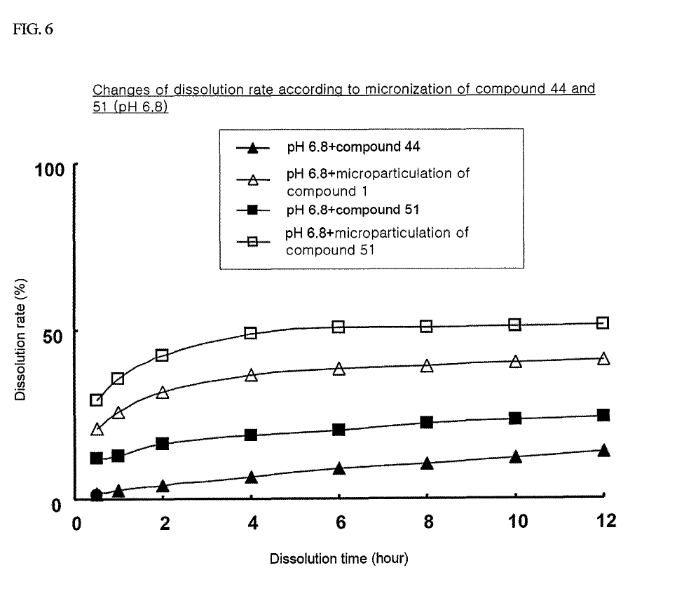

Further, changes of the dissolution rate were measured respectively at a different time points, and the results are provided in FIGS. 3 to 6.

Referring to FIGS. 3 to 6, the dissolution rates of the micronized particles of Compounds 1, 5, 58, 44 and 51 increased significantly comparing to those of before micronization, particularly, the dissolution rates of the micronized particles of Compounds 44 and 51 and Compound 58 Extract increased by more than 150%.

### Example 16

### Dissolution Rate of Micronized Particles of Compound 1 (β-Lapachon) and Dissolution Rate of Granulated Preparation

5.4 wt % of light anhydrous silicic acid, 30.4 wt % of Croscarmellose sodium, and 5.4 wt % of sodium lauryl sulfate were added to micronized particles of β-lapachon and then uniformly mixed. The mixture thus prepared was coated by 6.25% HPMC solution in ethanol and then granulated by 10% HPMC/ethanol solution.

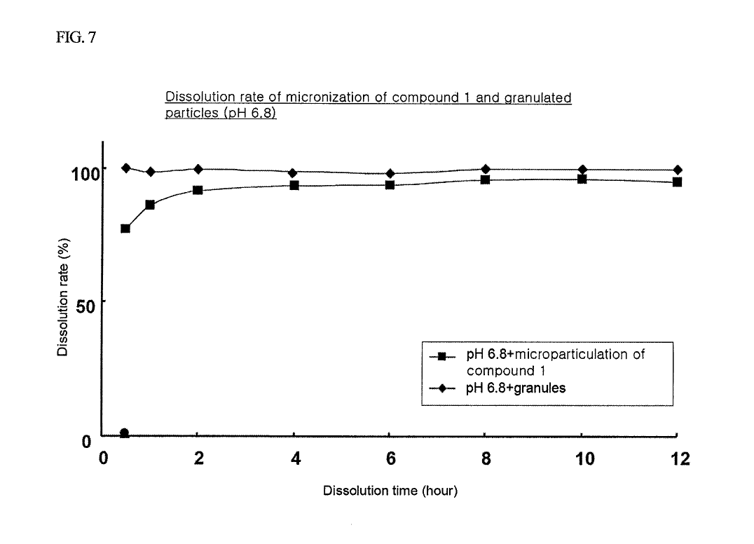

The dissolution rates of the granulated preparation of β-lapachon, as obtained above, and the micronized particles are provided in FIG. 7. The test for determining the dissolution rate was carried out in the same manner as in Example 15.

Referring to FIG. 7, it was ascertained that where the micronized particles are granulated using proper adjuvants, the dissolution rate until 12 hours is not significantly changed but the initial dissolution time is shortened.

### INDUSTRIAL APPLICABILITY

As apparent from the above description, the present invention can achieve improved availability and efficacy of an active drug by increasing bioavailability of naphthoquinone-based compounds via enhanced solubility and bioabsorption rate of the drug arising from stability and easy dispersibility in aqueous solutions or non-polar solvents, upon the incorporation of micronized particles of naphthoquinone-based compounds of Formula 1 or 2 as a sparingly-soluble substance having therapeutic effects on treatment and prevention of metabolic diseases. Further, since a composition comprising the micronized particles of naphthoquinone-based compound can be formulated into an oral preparation, the present invention provides an oral pharmaceutical preparation of naphthoquinone-based compounds for the first time.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A pharmaceutical composition comprising micronized particles of naphthoquinone-based compound, wherein the naphthoquinone-based compound is one or more selected from the compounds as represented by Formulas 1 and 2: 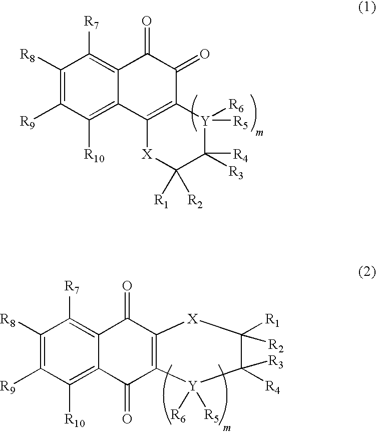
wherein
R₁ to R₆ are each independently hydrogen, hydroxyl, halogen, amino, alkylamino, dialkylamino, substituted or unsubstituted C₁-C₁₀ alkyl, substituted or unsubstituted C₁-C₁₀ alkenyl, substituted or unsubstituted C₁-C₁₀ alkynyl, substituted or unsubstituted C₁-C₁₀ alkoxy, substituted or unsubstituted C₁-C₁₀ alkoxycarbonyl, substituted or unsubstituted C₁-C₁₀ acyl, substituted or unsubstituted C₃-C₈ cycloalkyl, substituted or unsubstituted C₄-C₁₀ aryl, substituted or unsubstituted —(CH₂)ₙ-aryl, substituted or unsubstituted —(CH₂)ₙ-heterocyclic and substituted or unsubstituted —(CH₂)ₙ₋₁₀-phenyl, or two substituents thereof may be taken together to form a double bond, or substituted or unsubstituted C₃-C₆ cyclic structure which may be saturated or partially or completely unsaturated, wherein the substituent may be at least one selected from the group consisting of hydrogen, hydroxyl, C₁-C₁₀ alkyl, substituted or unsubstituted C₁-C₁₀ alkynyl, C₁-C₁₀ alkoxy, C₁-C₁₀ alkoxycarbonyl and C₁-C₁₀ alkylamino;
R₇ to R₁₀ are each independently hydrogen, hydroxyl, halogen, amino, alkylamino, dialkylamino, substituted or unsubstituted C₁-C₁₀ alkyl, substituted or unsubstituted C₁-C₁₀ alkenyl, substituted or unsubstituted C₁-C₁₀ alkynyl, substituted or unsubstituted C₁-C₁₀ alkoxy, substituted or unsubstituted C₁-C₁₀ alkoxycarbonyl, substituted or unsubstituted C₁-C₁₀ acyl, substituted or unsubstituted C₄-C₁₀ aryl, and substituted or unsubstituted —(CH₂)ₙ₋₁₀-phenyl, or two substituents thereof may be taken together to form a double bond, or substituted or unsubstituted C₃-C₆ cyclic structure which may be saturated or partially or completely unsaturated, wherein the substituent may be at least one selected from the group consisting of hydrogen, hydroxyl, halogen, C₁-C₁₀ alkyl, C₁-C₁₀ alkenyl, C₁-C₁₀ alkynyl, C₁-C₁₀ alkoxy, alkoxycarbonyl, C₁-C₁₀ alkylamino, C₃-C₈ cycloalkyl, C₃-C₈ hetero cycloalkyl, C₄-C₁₀ aryl and C₄-C₁₀ heteroaryl;
X is O, S or NR′, wherein R′ is hydrogen or C₁-C₆ alkyl;
Y is C, S, N, or O, with proviso that when Y is S or O, R₅ and R₆ are nothing and when Y is N, R₅ is hydrogen or C₁-C₆ lower alkyl and R₆ is nothing; and
m is 0 or 1, with proviso that when m is 0, carbon atoms adjacent to m form a cyclic structure via a direct bond, and n is 0˜10 integer,
or a pharmaceutically acceptable salt, solvate or isomer thereof.

2. The pharmaceutical composition according to claim 1, wherein X is O or S, and Y is C or O.

3. The pharmaceutical composition according to claim 1, wherein the compound of Formula 1 is the compound of Formula 1-1 or the compound of Formula 1-2. 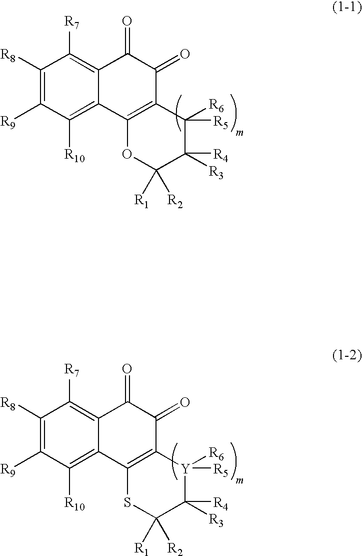
wherein, R₁ to R₁₀, Y and m are defined as in the claim 1.

4. The pharmaceutical composition according to claim 1, wherein the compound of Formula 1 is one of the compounds of Formula 1-3 to Formula 1-6. 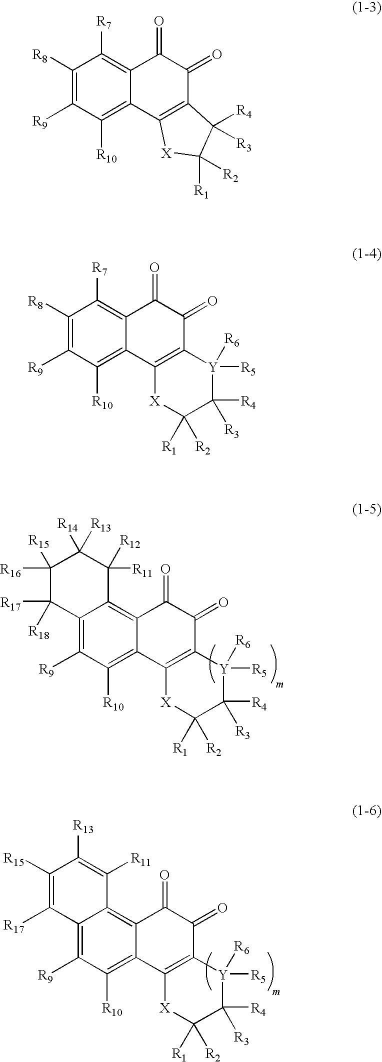
wherein, R₁ to R₁₈, X, Y and m are defined as in the claim 1.

5. The pharmaceutical composition according to claim 1, wherein R₁ to R₆ are each independently selected from the group consisting of hydrogen, hydroxy, halogen, substituted or unsubstituted C₁-C₁₀ alkyl, substituted or unsubstituted C₁-C₁₀ alkenyl, substituted or unsubstituted C₁-C₁₀ alkoxy and —(CH₂)ₙ-phenyl, or R₁ and R₂ or R₂ and R₃ are taken together to form a double bond, or substituted or unsubstituted C₃-C₆ cyclic structure, wherein the substituent is hydrogen or C₁-C₁₀ alkyl.

6. The pharmaceutical composition according to claim 1, wherein R₇ to R₁₀ are each independently selected from the group consisting of hydrogen, hydroxyl, halogen, substituted or unsubstituted C₁-C₁₀ alkyl, and substituted or unsubstituted C₁-C₁₀ alkoxy.

7. The pharmaceutical composition according to claim 1, wherein the naphthoquinone-based compound is selected from the compounds below: 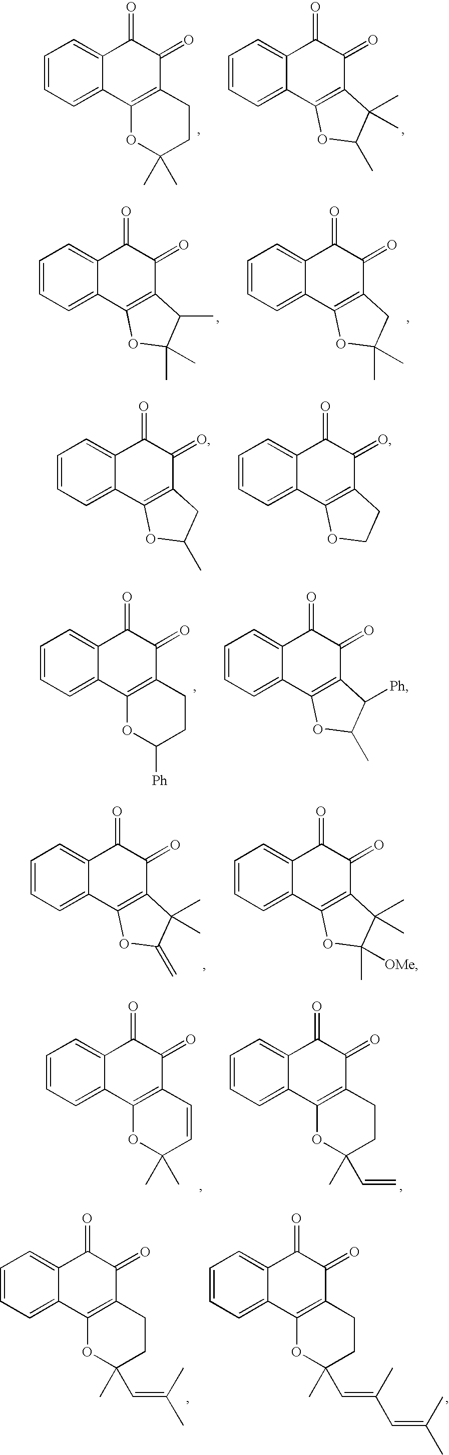 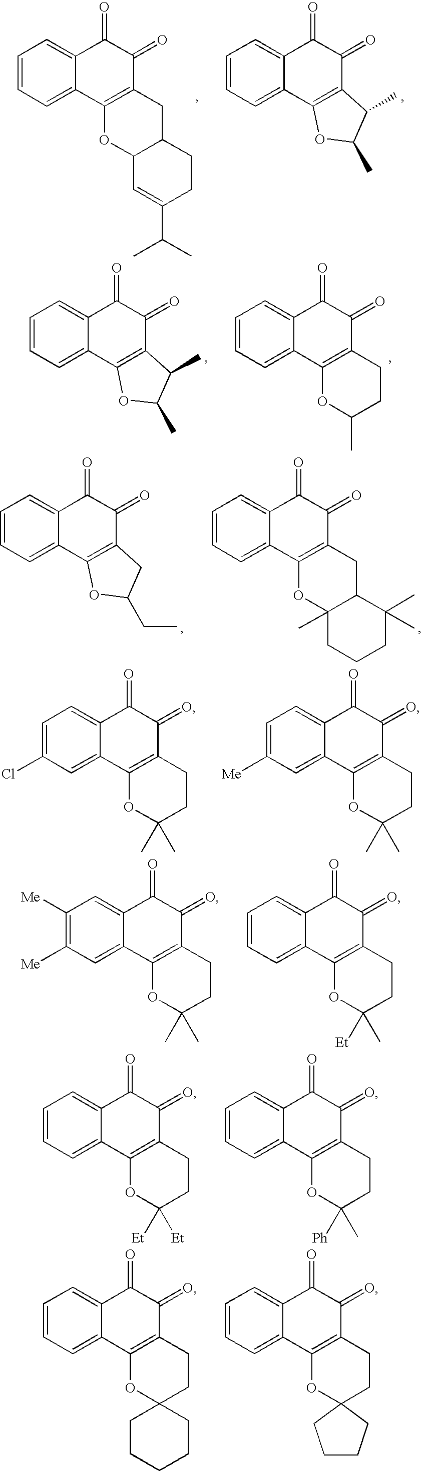 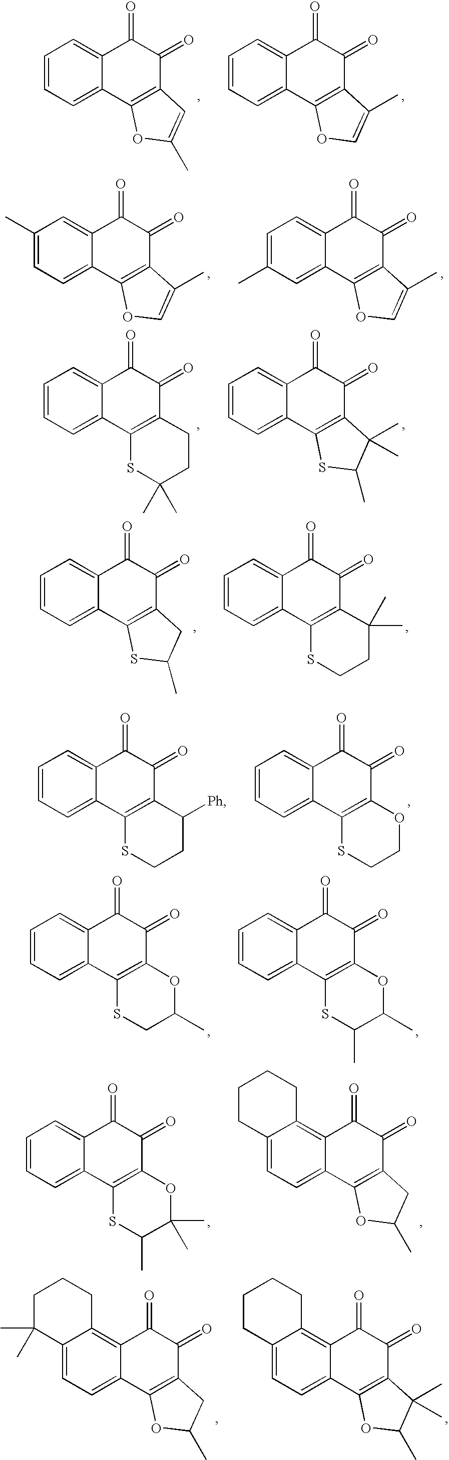 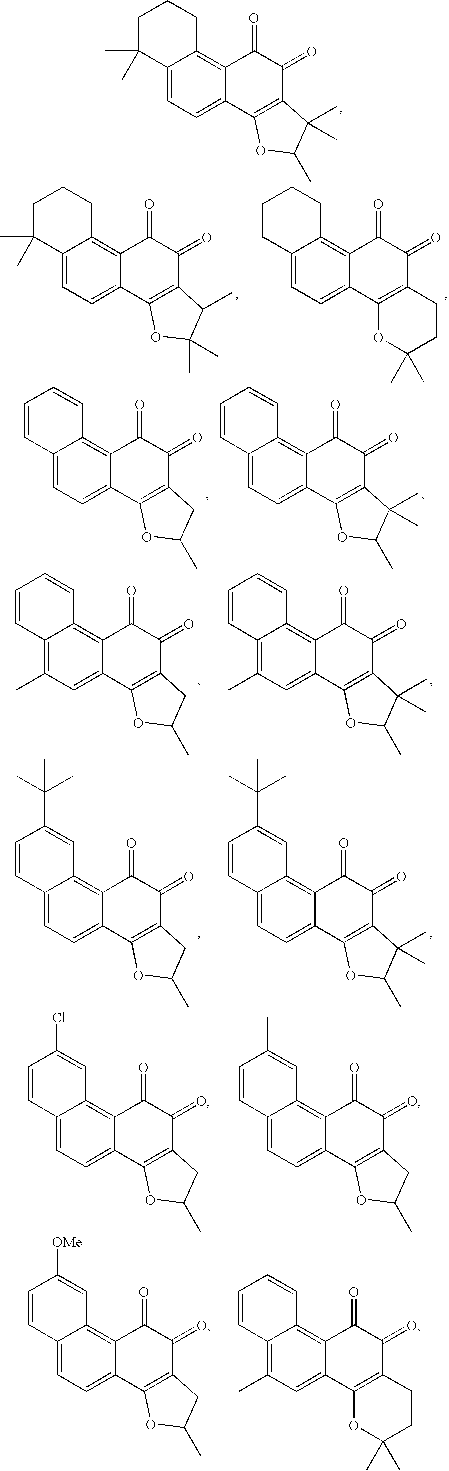 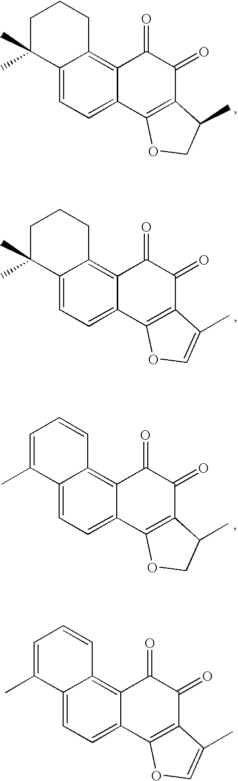
or a pharmaceutically acceptable salt, solvate or isomer thereof.

8. The pharmaceutical composition according to claim 1, wherein the size of micronized particles of the naphthoquinone-based compound is that 90% of the particle sizes (X90) is within the range of 1 nm to 30 μm.

9. The pharmaceutical composition according to claim 1, wherein the size of micronized particles of the naphthoquinone-based compound is that 50% of the particle sizes (X50) is within the range of 1 nm to 10 μm.

10. The pharmaceutical composition according to claim 1, wherein the pharmaceutical composition further comprises a surfactant.

11. The pharmaceutical composition according to claim 10, wherein the surfactant is contained in the range of 0.1 to 50% by weight based on the total weight of micronized particles of naphthoquinone-based compound and the surfactant.

12. The pharmaceutical composition according to claim 1, wherein the pharmaceutical composition further comprises a water-soluble polymer and/or a solubilizer.

13. The pharmaceutical composition according to claim 1, wherein the pharmaceutical composition further comprises a pharmaceutically acceptable vehicle and/or carrier.

14. The pharmaceutical composition according to claim 1, wherein the pharmaceutical composition is an oral pharmaceutical composition which is prepared into an intestine-targeted formulation.

15. The pharmaceutical composition according to claim 14, wherein the intestine-targeted formulation is prepared by addition of a pH-sensitive polymer.

16. The pharmaceutical composition according to claim 14, wherein the intestine-targeted formulation is prepared by addition of a biodegradable polymer which is decomposable by an intestine-specific bacterial enzyme.

17. The pharmaceutical composition according to claim 14, the intestine-targeted formulation is prepared by an addition of a biodegradable matrix which is decomposable by an intestine-specific bacterial enzyme.

18. The pharmaceutical composition according to claim 14, wherein the intestine-targeted formulation is constructed in a form which allows release of a drug after the pre-determined lag time.

19. The pharmaceutical composition according to claim 1, wherein the micronized particles of naphthoquinone-based compound are prepared by a micronization process which includes milling, precipitation, high-pressure homogenization, or supercritical micronization.

20. The pharmaceutical composition according to claim 1, wherein the micronized particles of naphthoquinone-based compound are administered at least over 10 mg per day.

21. The pharmaceutical composition according to claim 1, wherein the micronized particles of naphthoquinone-based compound are granulated.

22. The pharmaceutical composition according to claim 1, wherein the pharmaceutical composition is used for prevention and treatment of one or more selected from the group consisting of metabolic diseases, Restenosis, Impotence, prostatic disease, hypertension, cardiac diseases, renal diseases and glaucoma, degenerative diseases, and mitochondrial dysfunction-related diseases.

23. The pharmaceutical composition according to claim 22, wherein the metabolic syndrome is one or more selected from the group consisting of obesity, an obesity complication, a liver disease, arteriosclerosis, cerebral apoplexy, myocardial infarction, a cardiovascular disease, an ischemic disease, diabetes, a diabetes-related complication and an inflammatory disease.
